(19) United States
(12) Patent Application Publication
(10) Pub. No.: US 2025/0295711 A1
(43) Pub. Date: Sep. 25, 2025

(54) FOOD SUPPLEMENT

(71) Applicant: FONTERRA CO-OPERATIVE GROUP LIMITED, Auckland (NZ)

(72) Inventors: Aaron Calvin FANNING, Auckland (NZ); James William DEKKER, Auckland (NZ); Rolando SALTINI, Auckland (NZ); Xiaolei ZE, Auckland (NZ); Xuguang ZHANG, Auckland (NZ)

(73) Assignee: FONTERRA CO-OPERATIVE GROUP LIMITED, Auckland (NZ)

(21) Appl. No.: 18/832,675

(22) PCT Filed: Feb. 17, 2023
(86) PCT No.: PCT/IB2023/051434

§ 371 Date: Jul. 24, 2024

(30) Foreign Application Priority Data
Feb. 18, 2022 (EP) 22157612.7

(51) Int. Cl. A61K 35/745; A61K 35/747; A61P 25/22; A61P 25/24

(52) U.S. Cl. CPC A61K 35/745; A61K 35/747; A61P 25/22; A61P 25/24

(57) ABSTRACT

This invention relates to the use of probiotics for any one or more of (i) the prevention or reduction of stress; (ii) the reduction or prevention of anxiety; (iii) the prevention or reduction of one or more symptoms of depression; (iv) decreasing or reversing certain changes caused by stress, anxiety or depression; (v) improving mood; (vi) increasing relaxation; (vii) increasing energy, particularly to the use of Lacticaseibacillus rhamnosus strain HN001 (HN001) and/or Bifidobacterium animalis subsp. lactis strain HN019 (HN019).

### TECHNICAL FIELD

This invention relates to the use of probiotics, particularly Lactocaseibacillus rhamnosus strain HN001 (HN001) and/or Bifidobacterium animalis subsp. lactis strain HN019 (HN019) or derivatives thereof for any one or more of the following: (i) the prevention or reduction of stress; (ii) the prevention or reduction of anxiety; (iii) the prevention or reduction of one or more symptoms of depression; (iv) decreasing or reversing certain changes caused by stress, anxiety or depression; (v) improving mood; (vi) increasing relaxation; (vii) increasing energy. Methods for use of the bacteria and compositions and food products comprising the probiotic bacteria are also provided.

### BACKGROUND

Stress is the experience of emotional or physical tension in response to an event or thought process. Stress can have physical and mental manifestations, such as stomach ache, muscle tension, headache, rapid breathing, fast heartbeat, sweating, shaking, fatigue, change in appetite, dizziness, confusion, feelings of impending doom, panic or nervousness, difficulty concentrating, gut discomfort or disfunction, irrational anger and restlessness. Anxiety is often the result of stress, and relates to feelings of unease, worry, nervousness, fear or apprehension around the subject's current or future state.

Experience of stress and anxiety are very common and are not necessarily bad. Short term stress and anxiety can help overcome challenging or dangerous situations. However, repeated, chronic or overwhelming stress and anxiety can lead to serious health consequences, both physically and psychologically. For instance, chronic stress is associated with heart disease, high blood pressure, diabetes, depression and panic disorders.

It has been estimated that the recent Covid-19 pandemic has significantly increased the prevalence of stress and anxiety in the general population, with estimates as high as 30% of people suffering from defined stress, anxiety or depression. While various therapies are available for sufferers of stress and anxiety, there is a growing appreciation that nutrition may be able to ameliorate at least mild symptoms of stress and anxiety, without some of the side effects associated with drug interventions. Gut/brain axis research is providing reasons to believe that dietary components that may offer gut health benefits might also influence aspects of mental well-being.

There is an increasing amount of literature linking the health of the microbiota in the gut to brain chemistry and behaviour via multiple bi-directional pathways, including the immune system, neurotransmitter pathways, and a vast network of afferent and efferent nerves linking the gut to the central nervous system, suggesting that probiotic enhancement of gut microbiota may improve mood outcomes.

Microbiota-gut-brain axis mechanism encompasses a bidirectional relationship between the brain and gastrointestinal organs. Dysregulation of the microbiota-gut-brain axis has been actively revealed in the context of various psychiatric diseases such as neurodevelopmental disorders, schizophrenia, anxiety disorders, and depression. See: Younjung Lee and Yong-Ku Kim, 2021 (Current Psychiatry Reports, 23(5):22).

Zagórska et al., 2020 (Beneficial Microbes, 11(8):717-732) gathered literature concerning the potential effects of gut microbiota on psychiatric disorders through neural pathways comprising the ‘gut-brain axis’. In addition, the influence of probiotics and prebiotics and dairy-rich diets combined with the intake of probiotics and prebiotics on gut microbiota and the subsequent relationship with brain function was reviewed. The authors reported that not all probiotic-/prebiotic-/dairy-rich diet-based treatments exhibited a psychobiotic effect on the CNS.

Taylor and Holscher 2020 (Nutritional Neuroscience, 23(3):237-250) summarised findings from clinical studies using dietary intervention to improve depression, anxiety, or stress and the role the gastrointestinal microbiota may have in these disorders. The authors reported that probiotic consumption improved psychological or biological measures of depression, anxiety, or stress in individuals predisposed to a mood disorder. Probiotics suppressed biological markers of stress in healthy individuals in a strain-dependent manner.

Published PCT patent application, WO 2018/220429 A1, describes the use of Lactobacillus rhamnosus HN001 or derivatives thereof to treat or prevent postnatal depression and postnatal anxiety.

Published PCT patent application, WO 2018/002240 A1, described studies involving the maternal dietary supplementation of mice during pregnancy and lactation with a combination of the following probiotics: Lactobacillus rhamnosus LPR and Bifidobacterium longum BL999. Levels of synaptophysin were found to be higher in the mice group administered with the probiotics compared to the control group without probiotics. Synaptophysin was described in the application as being a protein that allows better adaptation during fearful situations, helping to modulate how fear is experienced during anxious situations and to prevent later fear in similar situations.

There is a continued need to provide compositions useful for reducing or preventing stress, anxiety and symptoms of depression, and generally for improving mood, increasing relaxation and for increasing energy levels. It would be particularly advantageous to be able to provide these benefits through nutrition, thereby avoiding potential side effects associated with drug interventions.

It is therefore an object of the present invention to address the aforementioned needs.

### SUMMARY OF THE INVENTION

The invention relates to the use of Lactocaseibacillus rhamnosus strain HN001 (HN001) and/or Bifidobacterium animalis subsp. lactis strain HN019 (HN019) or derivatives thereof for any one or more of the following: (i) the prevention or reduction of stress; (ii) the prevention or reduction of anxiety; (iii) the prevention or reduction of one or more symptoms of depression; (iv) decreasing or reversing certain changes caused by stress, anxiety or depression; (v) improving mood; (vi) increasing relaxation; (vii) increasing energy.

According to a further aspect of the invention, there is provided a composition comprising:
(i) Lactocaseibacillus rhamnosus strain HN001 (HN001) and/or derivatives thereof; and/or
(ii) Bifidobacterium animalis subsp. lactis strain HN019 (HN019) and/or derivatives thereof; and
(iii) a physiologically acceptable diluent, adjuvant, carrier or excipient,
   for use in any one or more of the following: (i) the reduction or prevention of stress; (ii) the reduction or prevention of anxiety; (iii) the prevention or reduction of one or more symptoms of depression; (iv) decreasing or reversing certain changes caused by stress, anxiety or depression; (v) improving mood; (vi) increasing relaxation; (vii) increasing energy.

A further aspect of the invention provides a method for any one or more of the following: (i) the reduction or prevention of stress; (ii) the reduction or prevention of anxiety; (iii) the prevention or reduction of one or more symptoms of depression; (iv) decreasing or reversing certain changes caused by stress, anxiety or depression; (v) improving mood; (vi) increasing relaxation; (vii) increasing energy, comprising administering Lactocaseibacillus rhamnosus strain HN001 (HN001) and/or Bifidobacterium animalis subsp. lactis strain HN019 (HN019) and/or derivatives thereof to a subject in need thereof. The method may be a non-therapeutic method.

Also provided is the use of Lactocaseibacillus rhamnosus strain HN001 (HN001) and/or Bifidobacterium animalis subsp. lactis strain HN019 (HN019) in the manufacture of a formulation for any one or more of the following: (i) the reduction or prevention of stress; (ii) for the reduction or prevention of anxiety; (iii) the prevention or reduction of one or more symptoms of depression; (iv) decreasing or reversing certain changes caused by stress, anxiety or depression; (v) improving mood; (vi) increasing relaxation; (vii) increasing energy.

A further aspect of the invention provides a food ingredient, food product or dietary supplement comprising, consisting of or essentially consisting of Lactocaseibacillus rhamnosus strain HN001 (HN001) and/or Bifidobacterium animalis subsp. lactis strain HN019 (HN019) for any one or more of the following: (i) the reduction or prevention of stress; (ii) the reduction or prevention of anxiety; (iii) the prevention or reduction of one or more symptoms of depression; (iv) decreasing or reversing certain changes caused by stress, anxiety or depression; (v) improving mood; (vi) increasing relaxation; (vii) increasing energy.

According to one embodiment, aspects of the invention relating to HN001 do not include or are not for the purpose of treatment or prevention of postnatal depression or postnatal anxiety.

According to one embodiment, aspects of the invention relating to HN001 do not include or are not for the purpose of enhancing calm or reducing occasional anxiety.

According to another embodiment, aspects of the invention relating to HN001, do not include the combination of Lactobacillus rhamnosus LPR (ATCC 54193) and Bifidobacterium longum BL999 (ATCC BAA-9999) for the purpose of reduction of anxiety and chronic stress.

According to another embodiment, aspects of the invention relating to HN001, do not include the combination of HN001 and glutamine and/or curcuma.

### BRIEF DESCRIPTION OF THE DRAWINGS

One or more embodiments of the invention will now be described, by way of example only, with reference to the accompanying drawings, in which:

FIG. 1 shows a schematic representation of the experimental set up using the CUMS (Chronic Unpredictable Mild Stress) model.

FIG. 2 shows an image of the open field experiment (left-hand image) and the elevated-plus maze experiment (right-hand image).

FIG. 3 shows the change in body weight of rat under CUMS, CUMS-HN001, CUMS-HN019, CUMS-HN019 and CUMS-Flu compared to controls.

FIG. 4 shows Western blot analysis of neuroendocrine hormone receptors corticotropin releasing factor type 1 (CRF1) and Glucocorticoid Receptor (GR) in brain tissue. Results shown as ratios of antibody staining intensity of receptor (CFR1, left; GR, right) vs the housekeeping protein GAPDH. * P<0.05.

FIG. 5 shows HE staining of brain and colon tissues. Brain tissues included two regions from the hippocampus (Dentate Gyrus (DG) and CA1) and a region from the cortex (P2).

FIG. 6 shows probiotic normalised CUMS-induced changes in neurotransmitters and their metabolite in rat serum and brain. The level of serum and brain 5-HT, DA, NE, Glu and GABA were detected at the end of the experiment (A, B, D, E, G, H, J, K, L). (C), (F) and (I) show the metabolites of monoamine neurotransmitters in brain. The results are presented as the mean±SEM (n=10). #P<0.05 compared with CUMS group.

FIG. 7 shows levels of IL-6, IL-18, IL-1β and TNF-α in serum, brain and colon of CUMS rats in different groups during experiment. These indicators were tested at the end of experiment. The results were presented as the mean±SEM (n=10). *P<0.05 compared with control group. #P<0.05 compared with CUMS group.

FIG. 8 shows four scatter plots of the correlation of TNF-α(A), IL-6(B), IL-1β(C) and IL-18(D) in brain and colon. ‘rs’ represent the spearman correlation coefficient.

FIG. 9 shows the effect of probiotic treatment on gut microbiota induced by CUMS. (A) Sobs index, (B) ACE index, (C) Chao1 index, (D) Shonnnon index. *P<0.05 compared with control group. #P<0.05 compared with CUMS group, & P<0.05 compared with CUMS-HN001&HN019 group.

FIG. 10 shows the analysis of microbiota flora composition among the six groups: control, CUMS, CUMS-HN001, CUMS-HN019, CUMS-HN001 & HN019 and CUMS-Flu. (A) unweighted PcoA, (B), (D) and (E) difference of microbiota flora abundance at the phylum, family and genus among the six groups. The area of each colour represents the abundance of different bacteria in the intestine. The larger the area, the higher the abundance, and vice versa. Data are stated as mean percentage values from each group (n=8 per group). (C) Comparison of the ratio of Firmicutes to Bacteroidetes among the six groups.

FIG. 11 shows a heat map showing the correlation between the phylum(A), family(B), genus(C) and monoamine neurotransmitters and Inflammatory factors. Red and blue colours indicate positive and negative correlations, respectively. *P<0.1, **P<0.05.

### DETAILED DESCRIPTION

According to one aspect of the invention, there is provided use of Lactocaseibacillus rhamnosus strain HN001 (HN001) and/or Bifidobacterium animalis subsp. lactis strain HN019 (HN019) or derivatives thereof for any one or more of the following: (i) the prevention or reduction of stress; (ii) for the prevention or reduction of anxiety; (iii) the prevention or reduction of one or more symptoms of depression; (iv) decreasing or reversing certain changes caused by stress, anxiety or depression; (v) improving mood; (vi) increasing relaxation; (vii) increasing energy.

The American Psychological Association (APA) defines stress as the physiological or psychological response to internal or external stressors. Stress involves changes affecting nearly every system of the body, influencing how people feel and behave. For example, it may be manifested by palpitations, sweating, dry mouth, shortness of breath, fidgeting, accelerated speech, augmentation of negative emotions (if already being experienced), and longer duration of stress fatigue. Severe stress is manifested by the general adaptation syndrome. By causing these mind-body changes, stress contributes directly to psychological and physiological disorder and disease and affects mental and physical health, reducing quality of life.

Anxiety is defined by the APA as an emotion characterised by apprehension and symptoms of tension in which an individual anticipates impending danger, catastrophe, or misfortune. The body often mobilises itself to meet the perceived threat: Muscles become tense, breathing is faster, and the heart beats more rapidly.

Both stress and anxiety are emotional responses, however stress is typically caused by an external trigger. The trigger can be short-term, such as a work deadline or a fight with a loved one, or long-term, such as being unable to work, discrimination, or chronic illness. Anxiety, on the other hand, is defined by persistent, excessive worries that do not go away even in the absence of a stressor.

### Stress

A reduction in stress as defined herein, and as defined by the American Psychological Association (APA), is taken to mean a reduction in any one or more of the following: stomach aches, muscle tension, headaches, rapid breathing, shortness of breath, fast heartbeat, palpitations, sweating, dry mouth, shaking, fatigue, fidgeting, accelerated speech, augmentation of negative emotions (if already being experienced), duration of stress fatigue, change in appetite, dizziness, confusion, feelings of impending doom, panic, nervousness, difficulty in concentrating, gut discomfort or disfunction, irritability, irrational anger and restlessness.

The stress may be a mild ‘everyday’ stress. The stress may be chronic stress, which is defined as the physiological or psychological response to a prolonged internal or external stressful event (i.e., a stressor). The stress may be an acute stress.

### Anxiety

A reduction in anxiety as defined herein, and as defined by the American Psychological Association (APA), is taken to mean a reduction in any one or more of the following: apprehension, feelings of impending danger, catastrophe, misfortune, unease, worry, nervousness, and fear or apprehension around the subject's current or future state.

### Calmness

Calmness is the mental state of peace of mind being free from agitation, excitement, or disturbance. It also refers to being in a state of serenity, tranquillity, or peace. Calmness can most easily occur for the average person during a state of relaxation, but it can also be found during much more alert and aware states.

### Depression

As defined herein, and as defined by the American Psychological Association (APA), major depressive disorder (depression) is a serious mood disorder typified by persistent feelings of sadness, emptiness, pessimism and/or low mood that negatively impacts daily life activities, including eating, and working. Symptoms include feelings of hopelessness, irritability, guilt, worthlessness, or helplessness. It can also include loss of interest or pleasure in leisure activities, decreased energy, fatigue, or restlessness, and can lead to difficulties with concentration and focus, memory, and decision making. Depression can also disrupt normal appetite and/or weight, and can result in suicide, or attempted suicide. In addition, depression can involve digestive discomfort and other digestive problems, and other aches or pains without a clear physical cause.

While postnatal depression (PND; also known as perinatal depression or postpartum depression) share a number of features with major depressive episodes, PND is typified by a series of features that makes it a separate entity from major depressive disorder, over and above the obvious association between PND and pregnancy/childbirth. For example, PND is associated with hormonal changes specific to the transition from pregnancy to the postpartum period. Animal model experiments have shown that rats given hormones to mimic this transition develop behavioural changes typical of PND. In addition, PND is associated with specific psychosocial stressors including infant temperament, and the stress associated with caring for a new-born infant. In contrast to typical patients with major depressive disorder, PND sufferers exhibit decreased activation of many neural regions, such as a blunted amygdala response to negative stimuli not related to the infant, or reduced emotional responses and regulation in response to their own infant's cries.

Lactocaseibacillus Rhamnosus Strain HN001 (HN001) and Bifidobacterium animalis Subsp. lactis Strain HN019 (HN019)

Lactocaseibacillus rhamnosus strain HN001 (HN001) and Bifidobacterium animalis subsp. lactis strain HN019 (HN019) are probiotics. The term “probiotic” is well known in the art and refers to microbial cell preparations or components of microbial cells with a beneficial effect on the health or well-being of the host. Means for determining the type of probiotic microorganism and its level of activity are well known in the art and include, for example, assessment using a PBMC cytokine secretion assay. The term “prebiotic” is also well known in the art and refers to food substances intended to promote the growth of probiotic bacteria in the intestines.

HN001 may be the only probiotic administered to or for administering to a subject. Alternatively, HN019 may be the only probiotic administered to or for administering to a subject. Alternatively, HN001 and HN019 may be the only prebiotics administered or for administering to a subject. HN001 and HN019 may be administered in combination, either sequentially, in any order, or combined. HN001 and/or HN019 may also be administered individually or in combination together with one or more further probiotics and/or with one or more prebiotics. Examples of suitable probiotics for co-administration include other strains of Lactocaseibacillus rhamnosus and/or Bifidobacterium animalis subsp. lactis, Lactobacillus acidophilus (for example, Lactobacillus acidophilus (LAVRI-A1), Lactobacillus reuteri (for example, Lactobacillus reuteri ATCC 55730) or Bifidobacterium lactis (for example, Bifidobacterium lactis strain BB12), Bifidobacterium bifidum, Bacillus coagulum, Saccharomyces boulardii, or combinations thereof. Examples of suitable prebiotics for co-administration include fructooligosaccharides, galactooligosaccharides, inulin, human milk oligosaccharides and combinations thereof.

As described in PCT publication No. WO 99/10476, HN001 is available under Australian Government Analytical Laboratories (AGAL), deposit number NM97/09514, dated 18 Aug. 1997, and HN019 is available under AGAL deposit number NM97/09513, dated 18 Aug. 1997. This Budapest Treaty-recognised depository is now no longer referred to as AGAL, but rather is referred to as the National Measurement Institute of Australia (NMIA), located at 1/153 Bertie Street, Port Melbourne, Victoria, Australia 3207.

HN001 is well known in the art and its various properties and identifying characteristics are described in, for example, WO 99/10476 and WO 2018/220429. HN019 is also well known in the art and its various properties and identifying characteristics are described in, for example, WO 99/10476. HN001 is also described in Slykerman et al., 2017 (EBioMedicine 24:159-165) and Tay et al., 2020 (Nutrients 12:3530).

HN001 and HN019 are both commercially available from New Zealand Milk Products (NZMP™), the ingredients and solutions brand of dairy co-operative, Fonterra. HN001 and HN019 are commercially available from NZMP, SureStart™, the early life nutrition brand, and from Nutiani™, the health and wellness solutions brand of dairy co-operative, Fonterra. HN001 is also available under name “LactoB HN001™” and “Nutiani HN001™”. HN019 is available under the name “BifidoB HN019™” and “Nutiani HN019™”.

HN001 and/or HN019 are preferably administered in a reproductively viable form. Additionally or alternatively, a proportion of the HN001 and HN019 may be killed, lysed, fractionated or attenuated.

Derivatives of HN001 and/or HN019

As used herein, the term “derivatives” of HN001 and of HN019 refers to mutants and homologues of HN001 and/or of HN019 or of killed or attenuated HN001 and/or HN019, such as but not limited to heat-killed, lysed, fractionated, pressure-killed, irradiated, and UV- or light-treated bacteria, and material derived from the bacteria, including but not limited to bacterial cell wall compositions, bacterial cell lysates, lyophilised bacteria, probiotic factors from the bacteria, and the like, wherein the derivative retains probiotic activity. Methods to produce such derivatives or one or more probiotic factors, and particularly derivatives suitable for administration to a subject (for example, in a composition) are well-known in the art.

### Subject

The “subject” of the invention is a mammal, preferably a human. The subject may also be a model animal, such as rat, mouse or guinea pig. The mammal may be a companion animal, such as a cat, dog or horse, or may be livestock such as pigs, cattle, horses, goats, sheep, deer, or poultry.

The subject may be a human infant, child or adult, an infant being under the age of 12 months, a child being between the age of 12 months and puberty and an adult being a human older than a child, for example, age sixteen or older. For example, the infant or child subject may be between 0-3 months, 4-5 months, 6-9 months, 10-14 months, 15-18 months, 19-24 months, 25-30 months, 31-42 months. The infant or child subject may be up to one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine or thirty days in age. The infant or child subject may be up to one, two, three, four, five, six, seven, eight, nine, ten, eleven, twelve, thirteen, fourteen, fifteen, sixteen, seventeen, eighteen, nineteen, twenty, twenty-one, twenty-two, twenty-three, or up to twenty-four months of age. The infant or child subject may be up to one year in age, or up to two years of age, or up to three years of age, or up to four years of age, or up to five years of age, or up to six years of age, or up to seven years of age, or up to eight years of age, or up to nine years of age, or up to ten years of age, or up to eleven years of age or up to twelve years of age, or up to thirteen years of age or up to fourteen years of age or of any age prior to the onset of puberty.

The subject may be a premature infant, meaning an infant born prior to 37 weeks gestational age. The subject may be pregnant or lactating in which case the subject may be the female in question or her offspring.

The subject of the invention may be an otherwise healthy individual, for example, as determined by the Apgar scoring system (The American College of Obstetricians and Gynecologists, No. 644, October 2015) in the case of a newborn, or through the use of standard growth curves, such as WHO Child Growth Standards (Acta Paediatrica, 2006, Suppl 450: 76-85) for older subjects. The WHO Child Growth Standards may be used to convert weight, length, and head circumference into weight-for-age, length-for-age, and head-circumference-for-age Z-scores. The subject of the invention may suitably be “full term”, meaning a baby born after the end of the 37th week of gestation. In general, a “healthy” subject does not include low birth weight infants, very low birth weight infants, and premature infants, “premature” being a baby born before the end of the 37th week of gestation. Although, the aforementioned does not apply once the previously premature or low birth weight subjects enter into the healthy range.

The reduction in stress, anxiety and depression, and improved mood, increased relaxation, and increased energy (i.e., the technical effects of the invention) are achieved by administering the probiotics HN001 and/or HN019 or derivatives thereof to a subject, for example, a subject in need thereof. “A subject in need” may self-determine as being in need due to exhibiting one or more of the symptoms of stress, anxiety or depression, as defined herein, or the subject may themselves feel in need of a boost in mood or to feel more relaxed or to have increased energy levels. Alternatively, “a subject in need” may be formally diagnosed by a medical practitioner as having stress, anxiety or depression. Alternatively, the technical effects of the invention may also be realised without the subject being recognised as being in need. In particular, administering the probiotics HN001 and/or HN019 or derivatives thereof to a subject can advantageously have preventative effects in developing symptoms of stress, anxiety or depression.

While various routes and methods of administration are contemplated, oral administration, such as in a suitable composition, is currently preferred. The term “oral administration” includes oral, buccal, enteral and intra-gastric administration.

Administration to a subject of HN001 and/or HN019 or derivatives thereof advantageously prevents or reduces stress, the term “stress” being as defined herein. The subject may self-diagnose as being stressed, due to exhibiting one or more symptoms of stress, as defined herein, or may be diagnosed by a medical practitioner as being stressed or having symptoms of stress or a stress disorder.

Administration to a subject of HN001 and/or HN019 or derivatives thereof advantageously prevents or reduces anxiety, the term “anxiety” being as defined herein. The subject may self-diagnose as having anxiety, due to exhibiting one or more symptoms of anxiety, as defined herein, or may be diagnosed by a medical practitioner as having anxiety or having symptoms associated with anxiety or an anxiety disorder.

Administration to a subject of HN001 and/or HN019 or derivatives thereof advantageously prevents or reduces of one or more symptoms of depression, as provided herein under the definition of “depression”. The subject may self-diagnose as having depression, due to exhibiting one or more symptoms of depression, as defined herein, or may be diagnosed with depression or as having symptoms associated with depression, by a medical practitioner.

A reduction in any one or more of stress, anxiety or in the symptoms of depression may be qualitatively or quantitatively determined depending on the symptom in question. The reduction of stress, anxiety or in the symptoms of depression may be self-determined, due to an amelioration in one or more of the symptoms of stress, anxiety or depression, or the reduction may be determined by a medical practitioner.

A reduction in any one or more of stress, anxiety or in the symptoms of depression may lead to any one or more of “improved mood”, “increased relaxation” and “increased energy”. Alternatively, the “improved mood”, “increased relaxation” and “increased energy” may be independent of any reduction in stress, anxiety or in the symptoms of depression.

An improvement in mood, feelings of increased relaxation and increased energy levels may be self-determined by a subject.

Administration of HN001 and/or HN019 or derivatives thereof advantageously decreases or reverses certain changes caused by stress, anxiety or depression, such as changes in any one or more of the following:
a) changes in levels or activity of neurotransmitters or their metabolites,
b) changes in levels or activity of stress hormones,
c) changes in levels or activity of inflammation factors,
d) morphological changes to brain tissue,
e) morphological changes to the colon,
f) changes to gut microbiota,
g) changes in weight.
   Changes in Levels or Activity of Neurotransmitters or their Metabolites

Administration of HN001 and/or HN019 or derivatives advantageously reverses stress-induced decreases in any one or more of serotonin (5-HT), dopamine (DA), and norepinephrine (NE) and/or increases the concentration of glutamate and/or GABA (for example in serum and/or brain tissue). HIAA, DOPAC and HVA are metabolites of 5-HT, DA and NE and are also typically increased in stressed individuals. Administration of HN001 and/or HN019 to individuals suffering from stress, anxiety or depression can suitably restore HIAA, DOPAC and HVA to levels found in non-stressed individuals.

### Changes in Levels or Activity of Stress Hormones

Administration of HN001 and/or HN019 or derivatives thereof advantageously reduces levels of one or more stress hormones, for example, molecules involved in the hypothalamic-pituitary-adrenal (HPA) axis, such as levels of any one or more of the following: corticotrophin-releasing hormone (CRH), adrenocorticotrophic hormone (ACTG), and levels of glucocorticoid hormones, such as cortisol. CRH is a hormone that is produced in the hypothalamus in the brain in response to many forms of stress (e.g., physical and emotional stress). Increased CRH production has been associated with Alzheimer's disease and major depression. Short term effects of CRH include appetite suppression, increased anxiety, and possibly inflammation.

The prevention or reduction of stress and/or anxiety in a subject through the use HN001 and/or HN019 or derivatives thereof may occur via the HPA axis.

The invention therefore further provides use of Lactocaseibacillus rhamnosus (HN001) and/or Bifidobacterium animalis subsp. lactis (HN019) or derivatives thereof for the reduction of levels of one or more molecules involved in the hypothalamic-pituitary-adrenal (HPA) axis, such as levels of any one or more of the following: corticotrophin-releasing hormone (CRH), adrenocorticotrophic hormone (ACTG), and levels of glucocorticoid hormones, such as cortisol.

### Changes in Levels or Activity of Inflammatory Factors

Inflammatory cytokines were found to increase in stressed subjects. Inflammatory cytokines showing elevated levels in stressed individuals included IL-6, TNF-α, IL-1β and IL-18. Administering Lactocaseibacillus rhamnosus (HN001) and/or Bifidobacterium animalis subsp. lactis (HN019) or derivatives thereof advantageously suppressed the rise of these inflammatory factors, thereby effectively reversing stress induced changes. The reduction in levels of inflammatory cytokines may suitably be found in serum, brain and colon samples.

Administration of HN001 and/or HN019 or derivatives advantageously reduces levels and/or activity of inflammatory cytokines, such as any one or more of TNF-alpha, IL-6, IL-1β and IL-18.

### Morphological Changes to Brain Tissue (Hippocampus) and Colon

Use according to the invention of HN001 and/or HN019 or derivatives advantageously protects and maintains epithelial cell barriers from damage and loss of integrity, for example, due to inflammation. The inflammation may be neuroinflammation (inflammation of the brain). Neuroinflammation is associated with a number of conditions including neurodegenerative disorders such as Alzheimer's Disease, Parkinson's disease, Huntington disease, encephalomyelitis, multiple sclerosis, dementia. Other conditions include vasculitis, stroke, delirium, declines in cognitive performance (for instance spatial memory, executive function, processing speed and other signs and symptoms of mild cognitive impairment (MCI)). Depression has also been associated with increased neuroinflammation. Deterioration or failures in the blood brain barrier may enable pathogenic infection of neural tissues leading to meningitis, encephalitis, or brain abscesses. Hence, strengthening of the blood brain barrier may be protective from brain infections.

Use according to the invention of HN001 and/or HN019 or derivatives thereof may suitably be used to strengthen the blood brain barrier (BBB), which in turn may protect against brain infections, for example, by preventing pathogenic infection of neural tissues which could lead to meningitis, encephalitis, or brain abscesses.

Use according to the invention of HN001 and/or HN019 or derivatives thereof may also suitably be used in the treatment of any one or more of the following: neurodegenerative disorders such as Alzheimer's Disease, Parkinson's disease, Huntington disease, multiple sclerosis, dementia; vasculitis; stroke; delirium; declines in cognitive performance (for instance spatial memory, executive function, processing speed and other signs and symptoms of mild cognitive impairment (MCI)).

Further advantageously, HN001 and/or HN019 or derivatives thereof may also be used to reverse the impact of stress and/or anxiety on brain or colon cell morphology and/or to protect brain or colon tissue from cell damage associated with stress and/or anxiety.

The administration of HN001 and/or HN019 or derivatives thereof may advantageously be used to protect or improve Blood Brain Barrier (BBB) integrity, which in turn could lead to reduced brain inflammation and/or improved brain function, and in protecting or improving gut barrier integrity.

The protection of gut barrier integrity may also help in the treatment or prevention of irritable bowel syndrome (IBS) and/or systemic inflammation.

The administration of HN001 and/or HN019 or derivatives thereof may advantageously also be used to improve digestive/gastrointestinal health (e.g., to reduce constipation or diarrhoea and/or to improve regularity).

Therefore, according to a further aspect of the invention, HN001 and/or HN019 or derivatives thereof may be used in a method of therapy, for example, in the treatment or prevention of any of the following: any one or more of the following: neurodegenerative disorders such as Alzheimer's Disease, Parkinson's disease, Huntington disease, multiple sclerosis, dementia; vasculitis; stroke; delirium; declines in cognitive performance (for instance spatial memory, executive function, processing speed and other signs and symptoms of mild cognitive impairment (MCI)); protect or improve Blood Brain Barrier (BBB) integrity, which in turn could lead to reduced brain inflammation and/or improved brain function, and in protecting or improving gut barrier integrity; treatment or prevention of irritable bowel syndrome (IBS) and/or systemic inflammation; and/or to improve digestive/gastrointestinal health. The method comprising administering a therapeutically effective amount of HN001 and/or HN019 or derivatives thereof to a subject in need thereof.

As used herein, “treat,” “treatment,” or “treating,” refers to administering or providing a composition for prophylactic and/or therapeutic purposes.

As used herein, the terms “prophylactic treatment,” “prevent,” or “preventing,” refer to treating a subject who does not yet exhibit symptoms of a disease or condition, but who is susceptible to, or otherwise at risk of, a particular disease or condition, whereby the treatment reduces the likelihood that the patient will develop the disease or condition.

### Changes to Gut Microbiota

Further advantageously, HN001 and/or HN019 or derivatives thereof may also be used to restore changes to gut microbiota caused by stress back to their non-stress profile, which may be in terms of abundance, (alpha and beta) diversity, richness, levels of dominant flora etc.

HN001 and/or HN019 administration may suitably restore changes in levels of any one or more of Firmicutes, Bacteroidetes, Verrucomicrobias, Proteobacteras and Actinobacteras back to levels found in non-stressed individuals.

HN001 and/or HN019 administration may suitably restore changes in levels of any one or more of Ruminococcaceae, Lactobacillaceae, Lachnospiraceae back to levels found in non-stressed individuals.

HN001 and/or HN019 administration may suitably restore changes in levels of any one or more of Lactobacillus, Eubacterium, Coprostanoligenes, Lachnospiraceae back to levels found in non-stressed individuals.

HN001 and/or HN019 administration may suitably restore the Firmicutes to Bacteroidetes ratio (F/B ratio) back to the ratio found in non-stressed individuals.

Alpha-diversity as measured by richness may be measured using Sobs index, Chao1 index and ACE index. Diversity may be measured using the Shannon index. Beta diversity may be measured using PCoA.

### Changes in Weight

Use according to the invention of HN001 and/or HN019 or derivatives thereof may also suitably be used to restore alterations in weight (either increases or decreases) caused by any of stress, anxiety or depression.

HN001 and/or HN019 may be administered in the form of a composition, which may comprise a physiologically acceptable diluent, adjuvant, carrier or excipient.

Therefore, according to another aspect of the invention, there is provided a composition comprising:
(i) Lacticaseibacillus rhamnosus strain HN001 (HN001) and/or derivatives thereof; and/or
(ii) Bifidobacterium animalis subsp. lactis strain HN019 (HN019) and/or derivatives thereof; and
(iii) a physiologically acceptable diluent, adjuvant, carrier or excipient,
   for use in any one or more of: (i) the reduction or prevention of stress; (ii) in the reduction or prevention of anxiety; (iii) the prevention or reduction of one or more symptoms of depression; (iv) decreasing or reversing certain changes caused by stress, anxiety or depression; (v) improving mood; (vi) increasing relaxation; (vii) increasing energy.

According to a further aspect of the invention, there is provided a method for any one or more of: (i) the reduction or prevention of stress; (ii) the reduction or prevention of anxiety; (iii) the prevention or reduction of one or more symptoms of depression; (iv) decreasing or reversing certain changes caused by stress, anxiety or depression; (v) improving mood; (vi) increasing relaxation; (vii) increasing energy, comprising administering the aforementioned composition or administering Lactocaseibacillus rhamnosus strain HN001 (HN001) and/or Bifidobacterium animalis subsp. lactis strain HN019 (HN019) or derivatives thereof to a subject.

A further aspect of the invention provides the use of Lactocaseibacillus rhamnosus strain HN001 (HN001) and/or Bifidobacterium animalis subsp. lactis strain HN019 (HN019) in the manufacture of a formulation for any one or more of: (i) the reduction or prevention of stress; (ii) the reduction or prevention of anxiety; (iii) the prevention or reduction of one or more symptoms of depression; (iv) decreasing or reversing certain changes caused by stress, anxiety or depression; (v) improving mood; (vi) increasing relaxation; (vii) increasing energy. Appropriate formulations may be prepared by those skilled in the art.

The use, compositions or methods of the invention may also be used for the reduction of levels of one or more molecules involved in the hypothalamic-pituitary-adrenal (HPA) axis, such as levels of any one or more of the following: corticotrophin-releasing hormone (CRH), adrenocorticotrophic hormone (ACTG), and levels of glucocorticoid hormones, such as cortisol and/or for the reduction of levels and/or activity of inflammatory cytokines, such as TNF-alpha and/or IL-6 cytokine.

The increase of CRH in response to stress is the first step in what is known as the Hypothalamus-Pituitary-Adrenal (HPA) axis. This is characterised by hypothalamic release of CRH (in response to stress), which then binds to CRH receptors on the pituitary gland, which in turn stimulates the release of adrenocorticotropic hormone (ACTH). ACTH leaves the brain via the circulatory system and binds to receptors on the adrenal cortex, stimulating the release of glucocorticoid hormones, such as cortisol in humans.

When blood cortisol concentrations reach a certain level, it exerts negative feedback on the release of CRH from the hypothalamus and ACTCH from the pituitary gland, returning the system to homeostasis. However, with repeated exposure to stressors, the system can become habituated with repeated and sustained HPA axis activation. Chronic HPA activation has a range of potential consequences including depression and anxiety disorders and immune dysfunction, as well as increased risk of type 2 diabetes, neurodegenerative disorders, and inflammatory disorders.

Advantageously, according to the present invention, the stress induced CRH response is blunted by HN001, HN019, and HN001+HN019. Furthermore, ACTH and Cortisol (other components of the HPA axis) are also reduced closer to non-stressed control levels through the administration of HN001, HN019, and HN001+HN019. These findings have been backed up by data on neurotransmitter expression in the brain. The stress-related increase of the cellular receptor for CRH was found to be blunted by the probiotics, whereas the stress related decrease in glucocorticoid receptor was reduced by the probiotics.

The composition preferably comprises a physiologically acceptable diluent, adjuvant, carrier or excipient, which may comprise food. Alternatively, HN001 and/or HN019 and/or derivatives thereof may be administered in a food product or dietary supplement.

A further aspect of the invention therefore also provides a food ingredient, food product or dietary supplement comprising, consisting of or essentially consisting of Lactocaseibacillus rhamnosus strain HN001 (HN001) and/or Bifidobacterium animalis subsp. lactis strain HN019 (HN019) for any one or more of: (i) the reduction or prevention of stress; (ii) the reduction or prevention of anxiety; (iii) the prevention or reduction of one or more symptoms of depression; (iv) decreasing or reversing certain changes caused by stress, anxiety or depression; (v) improving mood; (vi) increasing relaxation; (vii) increasing energy.

The food product may be any edible consumer product which is able to carry bacteria or a bacterial derivative. Examples of such consumer products include the following non-limiting examples: cultured milk, yoghurt, cheese, milk and yoghurt drinks, or milk powder, baby food, fruit juice, rice pudding, rusks, purees, cereals, porridge, confectionary products, reconstituted fruit products, snack bars, food bars, muesli bars, spreads, sauces, dips, sports supplements including dairy and non-dairy based sports supplements, food additives such as protein sprinkles, dietary supplement products including daily supplement tablets, weaning foods and yoghurts, and formulas such as maternal formula, in powder or liquid form, including hypoallergenic embodiments of such compositions.

The food product may be selected from a maternal supplement, a dietetic product, an infant formula, a follow-on formula, and a growing-up formula. The term “infant formula” as referred to herein is taken to mean a composition for infants aged between 0 days and 6 months old and a “follow-on formula” refers to a composition for infants aged 6 months to 1 year. The term “growing up formula” as used herein refers to compositions directed to infants and children aged 1 year upwards. Growing-up formula includes growing-up milk powders or GUMPs. It will be appreciated by those skilled in the art that the age ranges for the different compositions: “infant formula”, “follow-on formula” and “growing-up formula” can vary from child to child depending on the individual's development. The term “maternal formula” as used herein refers to a composition to be taken by pregnant or lactating women. The food product can also be a dietetic product, which refers to a product specially processed or formulated to satisfy particular dietary requirements which exist because of a particular physical or physiological condition and/or specific diseases and disorders and which are presented as such. The aforementioned products may be in liquid form as concentrates or ready-to-drink liquids or provided as powder concentrates.

Food products, such as dairy based drinks (e.g. milk drinks and yogurt drinks) will typically comprise a protein source (e.g. a dairy protein source), a lipid source, a carbohydrate source, in addition to the HN001 and/or HN019 or derivatives thereof.

The composition or food product or supplement may also include other nutrients, such as proteins, carbohydrates, vitamins, minerals, or amino acids. The composition may be in a form suitable for oral use, such as a tablet, a hard or soft capsule, an aqueous or oil suspension, or a syrup; or in a form suitable for parenteral use, such as an aqueous propylene glycol solution, or a buffered aqueous solution. The amount of the active ingredient in the nutraceutical composition depends to a large extent on a subject's specific need. The amount also varies, as recognised by those skilled in the art, dependent on administration route, and possible co-usage of other probiotic factors or probiotic agents.

The composition or food product or supplement of the invention may be provided as a capsule. Capsules may contain any standard pharmaceutically acceptable materials such as gelatin or cellulose. Tablets can be formulated in accordance with conventional procedures by compressing mixtures of the active ingredients with a solid carrier and a lubricant. Examples of solid carriers include starch and sugar bentonite. Active ingredients can also be administered in a form of a hard shell tablet or a capsule containing a binder, e.g., lactose or mannitol, a conventional filler, and a tabletting agent. Pharmaceutical compositions can also be administered via the parenteral route. Examples of parenteral dosage forms include aqueous solutions, isotonic saline or 5% glucose of the active agent, or other well-known pharmaceutically acceptable excipients. Cyclodextrins, or other solubilising agents well-known to those familiar with the art, can be utilized as excipients for delivery of the therapeutic agent.

Flavourants, colourants, and other additives, carriers or excipients, as would be well known to those skilled in the art, may also be included in the compositions or food products or supplements of the invention. It will be appreciated that a broad range of additives or carriers may be included in such compositions or food products, for example to improve or preserve bacterial viability or to increase therapeutic efficacy of HN001 and HN019 or of derivatives thereof. For example, additives such as surfactants, wetters, humectants, stickers, dispersal agents, stabilisers, penetrants, and so-called stressing additives to improve bacterial cell vigour, growth, replication and survivability (such as potassium chloride, glycerol, sodium chloride and glucose), as well as cryoprotectants such as maltodextrin, may be included. Additives may also include compositions which assist in maintaining microorganism viability in long term storage, for example unrefined corn oil, or “invert” emulsions containing a mixture of oils and waxes on the outside and water, sodium alginate and bacteria on the inside.

The composition or food product or supplement of the invention may comprise live HN001 and/or live HN019, preferably in a reproductively viable form and amount. Methods to produce such compositions are well-known in the art. The composition may comprise a carbohydrate source, such as a disaccharide including, for example, sucrose, fructose, glucose, or dextrose. Preferably the carbohydrate source is one able to be aerobically or anaerobically utilised by HN001 and/or HN019. The composition is preferably capable of supporting reproductive viability of HN001 and HN019 for a period greater than about two weeks, preferably greater than about one month, about two months, about three months, about four months, about five months, more preferably greater than about six months, most preferably at least about 2 years to about 3 years or more.

Methods to produce the compositions of the invention are well-known in the art, and may use standard microbiological and pharmaceutical practices.

In one embodiment, a composition useful herein comprises, consists essentially of, or consists of at least about 0.1, 0.2, 0.5, 1, 5, 10, 15, 20, 25, 30, 35, 40, 45, 50, 55, 60, 65, 70, 75, 80, 85, 90, 95, 99, 99.5, 99.8 or 99.9% by weight of HN001 and/or HN019 and/or derivatives thereof. Useful ranges may be selected between any of these foregoing values. For example, from about 0.1 to about 50%, from about 0.2 to about 50%, from about 0.5 to about 50%, from about 1 to about 50%, from about 5 to about 50%, from about 10 to about 50%, from about 15 to about 50%, from about 20 to about 50%, from about 25 to about 50%, from about 30 to about 50%, from about 35 to about 50%, from about 40 to about 50%, from about 45 to about 50%, from about 0.1 to about 60%, from about 0.2 to about 60%, from about 0.5 to about 60%, from about 1 to about 60%, from about 5 to about 60%, from about 10 to about 60%, from about 15 to about 60%, from about 20 to about 60%, from about 25 to about 60%, from about 30 to about 60%, from about 35 to about 60%, from about 40 to about 60%, from about 45 to about 60%, from about 0.1 to about 70%, from about 0.2 to about 70%, from about 0.5 to about 70%, from about 1 to about 70%, from about 5 to about 70%, from about 10 to about 70%, from about 15 to about 70%, from about 20 to about 70%, from about 25 to about 70%, from about 30 to about 70%, from about 35 to about 70%, from about 40 to about 70%, from about 45 to about 70%, from about 0.1 to about 80%, from about 0.2 to about 80%, from about 0.5 to about 80%, from about 1 to about 80%, from about 5 to about 80%, from about 10 to about 80%, from about 15 to about 80%, from about 20 to about 80%, from about 25 to about 80%, from about 30 to about 80%, from about 35 to about 80%, from about 40 to about 80%, from about 45 to about 80%, from about 0.1 to about 90%, from about 0.2 to about 90%, from about 0.5 to about 90%, from about 1 to about 90%, from about 5 to about 90%, from about 10 to about 90%, from about 15 to about 90%, from about 20 to about 90%, from about 25 to about 90%, from about 30 to about 90%, from about 35 to about 90%, from about 40 to about 90%, from about 45 to about 90%, from about 0.1 to about 99%, from about 0.2 to about 99%, from about 0.5 to about 99%, from about 1 to about 99%, from about 5 to about 99%, from about 10 to about 99%, from about 15 to about 99%, from about 20 to about 99%, from about 25 to about 99%, from about 30 to about 99%, from about 35 to about 99%, from about 40 to about 99%, and from about 45 to about 99%.

In one embodiment a composition, food product, ingredient or dietary supplement useful herein comprises, consists essentially of, or consists of at least about 0.001, 0.01, 0.05, 0.1, 0.15, 0.2, 0.3, 0.4, 0.5, 1, 2, 3, 4, 5, 6, 7, 8, 9, 10, 11, 12, 13, 14, 15, 16, 17, 18 or 19 grams of HN001 and/or HN019 or derivatives thereof. Useful ranges may be selected between any of the foregoing values. For example, from about 0.01 to about 1 gram, about 0.01 to about 10 grams, about 0.01 to about 19 grams, from about 0.1 to about 1 grams, about 0.1 to about 10 grams, about 0.1 to about 19 grams, from about 1 to about 5 grams, about 1 to about 10 grams, about 1 to about 19 grams, about 5 to about 10 grams, and about 5 to about 19 grams.

In one embodiment a composition or food product or supplement useful herein comprising HN001 and/or HN019 or derivatives thereof additionally comprises about 0.1, 0.5, 1, 5, 10, 15, 20, 25, 30, 35, 40, 45, 50, 55, 60, 65, 70, 75, 80, 85, 90, 95, 97, 99, or 99.9% by weight of fresh whole milk or a milk derivative and useful ranges may be selected between any of these foregoing values. For example, from about 0.1 to about 50%, from about 0.2 to about 50%, from about 0.5 to about 50%, from about 1 to about 50%, from about 5 to about 50%, from about 10 to about 50%, from about 15 to about 50%, from about 20 to about 50%, from about 25 to about 50%, from about 30 to about 50%, from about 35 to about 50%, from about 40 to about 50%, and from about 45 to about 50%. The milk derivative may be selected from recombined, powdered or fresh skim milk, recombined or reconstituted whole or skim milk powder, skim milk concentrate, skim milk retentate, concentrated milk, ultrafiltered milk retentate, milk protein concentrate (MPC), milk protein isolate (MPI), calcium depleted milk protein concentrate (MPC), low fat milk, low fat milk protein concentrate (MPC), casein, caseinate, milk fat, cream, butter, ghee, anhydrous milk fat (AMF), buttermilk, butter serum, beta serum, hard milk fat fractions, soft milk fat fractions, sphingolipid fractions, milk fat globular membrane fractions, milk fat globular membrane lipid fractions, phospholipid fractions, complex lipid fractions, colostrum, a colostrum fraction, colostrum protein concentrate (CPC), colostrum whey, an immunoglobulin fraction from colostrum, whey (including sweet whey, lactic acid whey, mineral acid whey, or reconstituted whey powder), whey protein isolate (WPI), whey protein concentrate (WPC), a composition derived from any milk or colostrum processing stream, a composition derived from the retentate or permeate obtained by ultrafiltration or microfiltration of any milk or colostrum processing stream, a composition derived from the breakthrough or adsorbed fraction obtained by chromatographic (including but not limited to ion and gel permeation chromatography) separation of any milk or colostrum processing stream, extracts of any of these milk derivatives including extracts prepared by multistage fractionation, differential crystallisation, solvent fractionation, supercritical fractionation, near critical fractionation, distillation, centrifugal fractionation, or fractionation with a modifier (e.g. soaps or emulsifiers), hydrolysates of any of these derivatives, fractions of the hydrolysates, and any combination of any two or more of these derivatives, including combinations of hydrolysed and/or non-hydrolysed fractions. It should be understood that the source of these derivatives may be milk or colostrum or a combination thereof.

It will be apparent that the concentration of HN001 and/or HN019 or derivatives thereof in a composition formulated for administration may be less than that in a composition formulated for, for example, distribution or storage, and that the concentration of a composition formulated for storage and subsequent formulation into a composition suitable for administration must be adequate to allow said composition for administration to also be sufficiently concentrated so as to be able to be administered at a therapeutically efficacious dose.

When used in combination with another therapeutic agent, the administration of a composition useful herein and the other therapeutic agent may be simultaneous or sequential. Simultaneous administration includes the administration of a single dosage form that comprises all components or the administration of separate dosage forms at substantially the same time. Sequential administration includes administration according to different schedules, preferably so that there is an overlap in the periods during which the composition useful herein and other therapeutic agent(s) are provided.

Suitable agents with which the compositions useful herein can be separately, simultaneously or sequentially administered include one or more probiotic agents, one or more prebiotic agents, one or more phospholipids, one or more gangliosides, other suitable agents known in the art, and combinations thereof. Useful prebiotics include galactooligosaccharides (GOS), short chain GOS, long chain GOS, fructooligosaccharides (FOS), human milk oligosaccharides (HMO), short chain FOS, long chain FOS, inulin, galactans, fructans, lactulose, and any mixture of any two or more thereof. Other useful agents may include dietary fibre such as a fully or partially insoluble or indigestible dietary fibre.

In one embodiment, a composition useful herein includes or is administered simultaneously or sequentially with milk components such as whey protein, whey protein fractions (including acidic or basic whey protein fractions or a combination thereof), glycomacropeptide, lactoferrin, iron-lactoferrin, a functional lactoferrin variant, a functional lactoferrin fragment, a vitamin D or calcium, or combinations thereof. Useful milk component-containing compositions include compositions such as a food, drink, food additive, drink additive, dietary supplement, nutritional product, medical food or nutraceutical. Milk fractions enriched for these components may also be employed.

It should be understood that the additional therapeutic agents listed above (both food based and pharmaceutical agents) may also be employed in a method according to the invention where they are administered separately, simultaneously or sequentially with a composition useful herein.

The composition, food product, ingredient or dietary supplement useful herein may further comprise a pharmaceutically acceptable carrier. The composition is or may be formulated as a food, drink, food additive, drink additive, dietary supplement, nutritional product, medical food, enteral feeding product, parenteral feeding product, meal replacement, cosmeceutical, nutraceutical, medicament, or pharmaceutical. The supplement comprising HN001 and/or HN019 or derivatives thereof may be in the form of a tablet, a caplet, a pill, a hard or soft capsule or a lozenge. The supplement comprising HN001 and/or HN019 or derivatives thereof may be in the form of a sachet, a powder, a dispensable powder, granules, a suspension, an elixir, a liquid, or any other form that can be added to food or drink, including for example water, milk or fruit juice. The composition or food product or supplement useful herein may further comprise one or more constituents (such as antioxidants), for example, to prevent or reduce degradation of the composition during storage or after administration. The composition or food product or supplement useful herein may include any edible consumer product which is able to carry bacteria or bacterial derivatives, including heat-killed, pressure-killed, lysed, UV- or light-treated, irradiated, fractionated or otherwise killed or attenuated bacteria. Examples of suitable edible consumer products include aqueous products, baked goods, confectionary products including chocolate, gels, ice creams, reconstituted fruit products, snack bars, food bars, muesli bars, spreads, sauces, dips, dairy products including yoghurts and cheeses, drinks including dairy and non-dairy based drinks, milk, milk powders, sports supplements including dairy and non-dairy based sports supplements, fruit juice, food additives such as protein sprinkles, dietary supplement products including daily supplement tablets, weaning foods and yoghurts, and formulas such as infant formula, follow-on formula, or growing-up formula, in powder or liquid form. Suitable nutraceutical compositions useful herein may be provided in similar forms.

It will be appreciated that different compositions of the invention may be formulated with a view to administration to a particular subject group. For example, the formulation of a composition suitable to be administered to a pregnant mother (for example, for indirect administration to a foetal subject or to a breastfeeding neonatal, infant, or child subject) may differ to that of a composition to be directly administered to the subject. It should also be appreciated that the formulation of a composition to be administered prophylactically, i.e., before symptoms appear, may differ to that of a composition formulated to a subject already exhibiting symptoms of stress and anxiety.

It is preferred that at least a part of the probiotic, is alive in the composition and preferably arrives alive in the intestine. This way they can persist in the intestines and may increase the effectiveness through multiplication. They may also be effective by interacting with the commensal bacteria and/or the host.

The compositions, food products, ingredients and supplements useful in the invention are administered in an amount sufficient for any one or more of the following: (i) the reduction or prevention of stress; (ii) the reduction or prevention of anxiety; (iii) the prevention or reduction of one or more symptoms of depression; (iv) decreasing or reversing certain changes caused by stress, anxiety or depression; (v) improving mood; (vi) increasing relaxation; (vii) increasing energy. An amount adequate to accomplish this is defined as “a therapeutically effective dose”. Amounts effective for this purpose will depend on a number of factors known to those skilled in the art, such as the severity of the stress, anxiety and the weight and general state of the patient.

In prophylactic applications, compositions, food products and supplements according to the invention are administered to a patient susceptible to or otherwise at risk of stress or anxiety, in an amount that is sufficient to at least partially reduce the risk of developing a disease. Such an amount is defined to be “a prophylactic effective dose”. Again, the precise amounts depend on a number of patient-specific factors such as a patient's state of health and weight.

Generally, the probiotic contained in the composition, food product or supplement according to the invention will be administered in a therapeutically effective dose and/or in a prophylactic effective dose.

If said probiotic is present in a viable form, it is theoretically effective at any concentration considering that bacteria can colonise the gut and multiply.

These expressions of quantity of bacteria include the possibilities that the bacteria are live, inactivated or dead or even present as fragments such as DNA or cell wall materials or as metabolites. In other words, the quantities of bacteria are expressed in terms of the colony forming ability (cfu) of that quantity of bacteria as if all the bacteria were live irrespective of whether they are, in fact, live, inactivated or dead, fragmented or a mixture of any or all of these states.

HN001 and/or HN019 or derivatives thereof, or the food product or supplement comprising the same, may be administered daily. Daily administration refers to the quantity to be taken in a day (24 h), by means of one or several doses.

An oral composition is formulated to allow the administration of a sufficient amount of HN001 and/or HN019 or derivatives thereof to establish a population in the gastrointestinal tract of the subject when ingested. The population established may be transient or permanent.

In theory, one colony forming unit (cfu) should be sufficient to establish a population of HN001 and/or HN019 in a subject, however for effects that are reliant on a viable, living population of the probiotic bacteria, a greater the number of units administered to a subject will improve efficacy.

The Applicants have determined that a dosage rate of 1.9×10⁹ cfu of HN001 per kg body weight per day and/or 6.5×10⁷ cfu of HN019 per kg body weight day is sufficient to establish a population in the gastrointestinal tract of human subjects. Accordingly, in one example, a composition formulated for administration will be sufficient to provide at least about 1.9×10⁹ cfu of HN001 and/or 6.5×10⁷ cfu of HN019 and/or derivatives thereof per kg body weight per day.

Methods to determine the presence of a population of gut flora, such as of HN001 and/or HN019, in the gastrointestinal tract of a subject are well known in the art. In certain embodiments, presence of a population of HN001 and/or HN019 may be determined directly, for example by analysing one or more samples obtained from a subject and determining the presence or amount of HN001 and/or HN019 in said sample. In other embodiments, presence of a population of HN001 and/or HN019 may be determined indirectly, for example by observing any one or more of: (i) the reduction or prevention of stress; (ii) the reduction or prevention of anxiety; (iii) the prevention or reduction of one or more symptoms of depression; (iv) decreasing or reversing certain changes caused by stress, anxiety or depression; (v) improving mood; (vi) increasing relaxation; (vii) increasing energy. Combinations of such methods are also envisaged.

The efficacy of a composition useful in the invention may be evaluated both in vitro and in vivo. For example, the composition may be tested for its ability to do any one or more of: (i) reduce or prevent stress; (ii) reduce or prevent anxiety; (iii) prevent or reduce of one or more symptoms of depression; (iv) decrease or reverse certain changes caused by stress, anxiety or depression; (v) improving mood; (vi) increasing relaxation; (vii) increasing energy. For in vivo studies, the composition may be fed to or injected into an animal model (e.g., a mouse) or administered to human subjects and its effect on any of features (i) to (v) above can then be assessed. Based on the results, an appropriate dosage range and administration route can be determined.

Methods of calculating appropriate dose may depend on the nature of the active agent in the composition. For example, when the composition comprises live HN001 and/or HN019, the dose may be calculated with reference to the number of live bacteria present. For example, the dose may be established by reference to the number of colony forming units (cfu) to be administered per day. In examples where the composition comprises one or more HN001 and/or HN019 derivatives, the dose may be calculated by reference to the amount or concentration of HN001 and/or HN019 derivative present. For example, for a composition comprising HN001 and/or HN019 cell lysate, the dose may be calculated by reference to the concentration of HN001 and/or HN019 cell lysate present in the composition.

By way of general example, the administration may be in the range of from about 1×10⁶ cfu to about 1×10¹² cfu of HN001 and/or HN019 per kg body weight per day, such as about 1×10⁶ cfu to about 1×10¹¹ cfu/kg/day; about 1×10⁶ cfu to about 1×10¹⁰ cfu/kg/day; about 1×10⁶ cfu to about 1×10⁹ cfu/kg/day; about 1×10⁶ cfu to about 1×10⁸ cfu/kg/day; or about 1×10⁶ cfu to about 1×10⁷ cfu/kg/day is contemplated. The administration may be from about 5×10⁶ cfu to about 4×10⁸ cfu/kg/day; about 5×10⁶ cfu to about 3×10⁸ cfu/kg/day; about 5×10⁶ cfu to about 2×10⁸ cfu/kg/day; about 5×10⁶ cfu to about 1×10⁸ cfu/kg/day; about 5×10⁶ cfu to about 9×10⁷ cfu/kg/day; about 5×10⁶ cfu to about 8×10⁷ cfu/kg/day; about 5×10⁶ cfu to about 7×10⁷ cfu/kg/day; about 5×10⁶ cfu to about 6×10⁷ cfu/kg/day; about 5×10⁶ cfu to about 5×10⁷ cfu/kg/day; about 5×10⁶ cfu to about 4×10⁷ cfu/kg/day; about 5×10⁶ cfu to about 3×10⁷ cfu/kg/day; about 5×10⁶ cfu to about 2×10⁷ cfu/kg/day; or about 5×10⁶ cfu to about 1×10⁷ cfu/kg/day, is contemplated.

In certain embodiments, periodic dose need not vary with body weight or other characteristics of the subject. In such examples, the administration may be from about 1×10⁶ cfu to about 1×10¹³ cfu of HN001 and/or HN019 per day, preferably about 1×10⁶ cfu to about 1×10¹² cfu/day; about 1×10⁶ cfu to about 1×10¹¹ cfu/day; about 1×10⁶ cfu to about 1×10¹⁰ cfu/day; about 1×10⁶ cfu to about 1×10¹¹ cfu/day; about 1×10⁶ cfu to about 1×10⁸ cfu/day; about 1×10⁶ cfu to about 1×10⁷ cfu/day is contemplated. The administration may be from about 2×10⁶ cfu to about 2×10¹³ cfu of HN001 and/or HN019 per day, preferably about 2×10⁶ cfu to about 2×10¹² cfu/day; about 2×10⁶ cfu to about 2×10¹¹ cfu/day; about 2×10⁶ cfu to about 2×10¹⁰ cfu/day; about 2×10⁶ cfu to about 2×10⁹ cfu/day; about 2×10⁶ cfu to about 2×10⁸ cfu/day; about 2×10⁶ cfu to about 2×10⁷ cfu/day is contemplated. The administration may be from about 3×10⁶ cfu to about 3×10¹³ cfu of HN001 and/or HN019 per day, preferably about 3×10⁶ cfu to about 3×10¹² cfu/day; about 3×10⁶ cfu to about 3×10¹¹ cfu/day; about 3×10⁶ cfu to about 3×10¹⁰ cfu/day; about 3×10⁶ cfu to about 3×10⁹ cfu/day; about 3×10⁶ cfu to about 3×10⁸ cfu/day; about 3×10⁶ cfu to about 3×10⁷ cfu/day is contemplated. The administration may be from about 4×10⁶ cfu to about 4×10¹³ cfu of HN001 and/or HN019 per day, preferably about 4×10⁶ cfu to about 4×10¹² cfu/day; about 4×10⁶ cfu to about 4×10¹¹ cfu/day; about 4×10⁶ cfu to about 4×10¹⁰ cfu/day; about 4×10⁶ cfu to about 4×10⁹ cfu/day; about 4×10⁶ cfu to about 4×10⁸ cfu/day; about 4×10⁶ cfu to about 4×10⁷ cfu/day is contemplated. The administration may be from about 5×10⁶ cfu to about 5×10¹³ cfu of HN001 and/or HN019 per day, preferably about 5×10⁶ cfu to about 5×10¹² cfu/day; about 5×10⁶ cfu to about 5×10¹¹ cfu/day; about 5×10⁶ cfu to about 5×10¹⁰ cfu/day; about 5×10⁶ cfu to about 5×10⁹ cfu/day; about 5×10⁶ cfu to about 5×10⁸ cfu/day; about 5×10⁶ cfu to about 5×10⁷ cfu/day is contemplated. The administration may be from about 6×10⁶ cfu to about 6×10¹³ cfu of HN001 and/or HN019 per day, preferably about 6×10⁶ cfu to about 6×10¹² cfu/day; about 6×10⁶ cfu to about 6×10¹¹ cfu/day; about 6×10⁶ cfu to about 6×10¹⁰ cfu/day; about 6×10⁶ cfu to about 6×10⁹ cfu/day; about 6×10⁶ cfu to about 6×10⁸ cfu/day; about 6×10⁶ cfu to about 6×10⁷ cfu/day is contemplated. The administration may be from about 7×10⁶ cfu to about 7×10¹³ cfu of HN001 and/or HN019 per day, preferably about 7×10⁶ cfu to about 7×10¹² cfu/day; about 7×10⁶ cfu to about 7×10¹¹ cfu/day; about 7×10⁶ cfu to about 7×10¹⁰ cfu/day; about 7×10⁶ cfu to about 7×10⁹ cfu/day; about 7×10⁶ cfu to about 7×10⁸ cfu/day; about 7×10⁶ cfu to about 7×10⁷ cfu/day is contemplated.

Preferably, the administration is from about 7×10⁷ cfu to about 7×10¹⁰ 5×10⁷ cfu; to about 5×10¹⁰ cfu per kg body weight of HN001 and/or HN019 per day, preferably about 5×10⁷ cfu to about 4×10¹⁰ cfu/day; about 5×10⁷ cfu to about 3×10¹⁰ cfu/day; about 5×10⁷ cfu to about 2×10¹⁰ cfu/day; about 5×10⁷ cfu to about 1×10¹⁰ cfu/day; about 5×10⁷ cfu to about 9×10⁹ cfu/day; about 5×10⁷ cfu to about 8×10⁹ cfu/day; about 5×10⁷ cfu to about 7×10⁹ cfu/day; about 5×10⁷ cfu to about 6×10⁹ cfu/day; about 5×10⁷ cfu to about 5×10⁹ cfu/day; about 5×10⁷ cfu to about 4×10⁹ cfu/day; about 5×10⁷ cfu to about 3×10⁹ cfu/day; about 5×10⁷ cfu to about 2×10⁹ cfu/day; or about 5×10⁷ cfu to about 1×10⁹ cfu/day, is contemplated.

An efficacious dose of freeze-dried HN001 and/or HN019 was determined to be about 6×10⁹ cfu per day.

It will be appreciated that the composition is preferably formulated so as to allow the administration of an efficacious dose of HN001 and/or HN019 or one or more derivatives thereof. The dose of the composition administered, the period of administration, and the general administration regime may differ between subjects depending on such variables as the severity of symptoms of a subject, the type of disorder to be treated, the mode of administration chosen, and the age, sex and/or general health of a subject. Furthermore, as described above the appropriate dose may depend on the nature of the active agent in the composition and the manner of formulation. For example, when the composition comprises live HN001 and/or HN019, the dose may be calculated with reference to the number of live bacteria present. For example, as described herein the examples the dose may be established by reference to the number of colony forming units (cfu) to be administered per day. In examples where the composition comprises one or more HN001 and/or HN019 derivatives, the dose may be calculated by reference to the amount or concentration of HN001 and/or HN019 derivative to be administered per day. For example, for a composition comprising HN001 and/or HN019 cell lysate, the dose may be calculated by reference to the concentration of HN001 and/or HN019 cell lysate present in the composition.

It will be appreciated that preferred compositions are formulated to provide an efficacious dose in a convenient form and amount. In certain embodiments, such as but not limited to those where the periodic dose need not vary with body weight or other characteristics of the subject, the composition may formulated for unit dosage. It should be appreciated that administration may include a single daily dose or administration of a number of discrete divided doses as may be appropriate. For example, as presented herein in the examples, an efficacious dose of HN001 and/or HN019 may be formulated into a capsule for oral administration.

The composition, food product or supplement may be intended for use by humans, pets or livestock. The terms “intended for use by” means that there are specific adaptations for the nutritional needs of the targeted mammalian population. The skilled person is aware of the ingredients useful for such nutritional compositions, in addition to the probiotic according to the invention, so as to make them suitable as supplements or nutritionally complete composition.

According to one embodiment, aspects of the invention, as they relate to HN001, do not include the treatment or prevention of postnatal depression or postnatal anxiety.

According to one embodiment, aspects of the invention, as they relate to HN001, do not include enhancing calm or reducing occasional anxiety.

According to another embodiment, aspects of the invention, as they relate to HN001, do not include the combination of Lactobacillus rhamnosus LPR (ATCC 54193) and Bifidobacterium longum BL999 (ATCC BAA-9999) for the reduction of anxiety and chronic stress.

According to another embodiment, aspects of the invention relating to HN001, do not include the combination of HN001 and glutamine and/or curcuma.

Various aspects of the invention will now be illustrated in non-limiting ways by reference to the following examples.

### EXAMPLES

### Example 1: Chronic Unpredictable Mild Stress (CUMS) Model for Testing the Effects of Lacticaseibacillus rhamnosus (HN001) and Bifidobacterium animalis subsp. lactis (HN019)

A series of experiments were conducted using the chronic unpredictable mild stress (CUMS) rodent model. Rats were exposed daily to any two of the following mild physiological stresses selected at random:
12 hours of water deprivation
12 hours of food deprivation
8 hours of damp sawdust
24 hours of reversed light/dark cycle
2 minutes of tail pinching
1 hour of 60 Hz noise
15 minutes of cage shaking
Swimming in 4° C. water for 5-10 minutes
2 hours of restraining.

Such exposure induces depressive symptoms resulting in a variety of physiological effects and behaviours in the rats that are regarded as approximating anxiety and depression in humans.

Six groups of rats were assessed:
1. Control rats: rats raised normally without stress treatments and without nutritional or drug interventions.
2. CUMS rats: rats subjected to the CUMS protocol but without nutritional or drug interventions.
3. CUMS HN001: rats subjected to the CUMS protocol and given HN001.
4. CUMS HN019: rats subjected to the CUMS protocol and given HN019.
5. CUMS HN001+HN019: rats subjected to the CUMS protocol given both HN001 and HN019.
6. CUMS Fluox: rats subjected to the CUMS protocol and given fluoxetine, a type of antidepressant known as a selective serotonin reuptake inhibitor (SSRI), noted as having fewer unwanted effects than other antidepressants.

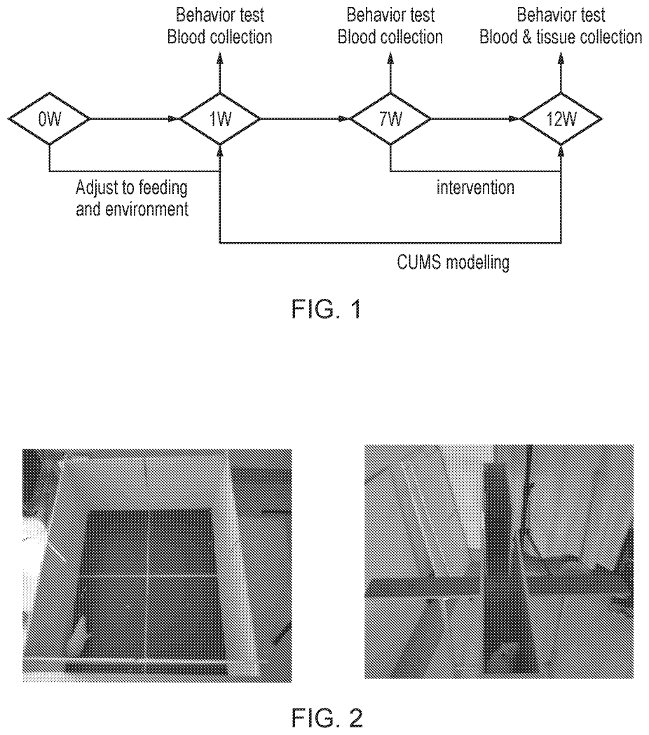

As shown in FIG. 1, experiments were conducted in which groups of rats (n=20 each) were acclimatised for seven days. Baseline assessments were then conducted, including blood collection. For the next 40 days, rats in groups 2-6 were exposed to CUMS treatments to induce depression symptoms associated with stress and anxiety. At this point, assessments and blood collection were repeated in all groups, after which groups 2-6 resumed the CUMS treatments with the addition of probiotic or fluoxetine interventions for a further 40 days.

Tests were then carried out to assess rat behaviours, such as the open field test and the elevated maze test (see FIG. 2). These are standard ways to measure rat stress and anxiety traits.

Stressed rats tended to be timid, avoiding open and lit spaces. Rats that had undergone CUMS showed decreased exploratory behaviours in both the open field and elevated maze tests, but these responses were largely reversed in CUMS rats that had been fed HN001, HN019, or both, to a similar level as rats that had been fed an anti-depressant drug, and almost to the same level as control rats that had not undergone CUMS.

### Results

### Example 2: Weight

CUMS-exposed rat groups showed reduced weight gain in comparison to control rats not exposed to CUMS, indicating that CUMS treatments did affect rat physiology. The reduced weight gain in rats exposed to CUMS was improved by administration of HN001, HN019, or both (FIG. 3). Also, there were no differences in weight gain between CUMS groups.

### Example 3: Open Field Test

In the first behavioural test—the open field test—all CUMS rats after the initial six week period (CUMS-treated but no probiotic/drug intervention) showed reduced activity, indicating that the rats were too anxious to explore their local environment; this is the expected response to CUMS. However, after the probiotic/drug intervention, while the rats exposed to CUMS, but not probiotics or fluoxetine, continued to have low activity in the open field test, all three probiotic groups showed increased activity levels similar to the control group, and similar to the CUMS rats treated with the anti-depressant fluoxetine. See tables below.

TABLE 1
Effects of CUMS and Intervention on Total Distance
Travelled (in Metres) in Open Field Test

| | | | After CUMS + |
|---|---|---|---|
| | Base line | After CUMS only | intervention |
| Group | (x̅ ± S) | (x̅ ± S) | (x̅ ± S) |
| Control | 20.22 ± 3.58 | 15.00 ± 5.66 | 14.59 ± 3.64^{b} |
| CUMS | 23.71 ± 6.35 | 7.40 ± 4.18^{a} | 4.01 ± 1.97 |
| CUMS-Flu | 23.34 ± 2.98 | 2.89 ± 2.15^{a} | 10.89 ± 4.02^{b} |
| CUMS-HN001 | 23.43 ± 8.42 | 5.64 ± 2.10^{a} | 14.62 ± 7.97^{b} |
| CUMS-HN019 | 24.65 ± 3.59 | 5.42 ± 3.11^{a} | 12.21 ± 2.54^{b} |
| CUMS-HN001 & | 22.94 ± 6.20 | 2.99 ± 2.04^{a} | 12.77 ± 4.67^{b} |
| HN019 | | | |

^{a}Compared to control, p < 0.05;
^{b}Compared to CUMS only, p < 0.05

TABLE 2
Effect of CUMS and Intervention on Vertical Movement
(Number of Times Standing Up) in Open Field Test

| | | | After |
|---|---|---|---|
| | Base line | After CUMS | intervention |
| Group | (x̅ ± S) | (x̅ ± S) | (x̅ ± S) |
| Control | 15.33 ± 8.21 | 14.00 ± 6.96 | 13.89 ± 10.65^{b} |
| CUMS | 15.67 ± 4.63 | 4.33 ± 3.14^{a} | 3.16 ± 3.13 |
| CUMS-Flu | 17.20 ± 8.04 | 2.00 ± 1.79^{a} | 11.20 ± 3.49^{b} |
| CUMS-HN001 | 19.67 ± 6.26 | 3.43 ± 2.88^{a} | 13.57 ± 4.39^{b} |
| CUMS-HN019 | 19.62 ± 6.80 | 4.50 ± 3.42^{a} | 11.63 ± 4.90^{b} |
| CUMS- | 19.75 ± 8.68 | 4.43 ± 3.82^{a} | 12.38 ± 4.14^{b} |
| HN001&HN019 | | | |

^{a}Compared to control, p < 0.05;
^{b}Compared to CUMS only, p < 0.05

### Example 4: Elevated-Plus Maze

Similar results were obtained using the second behavioural test—the elevated-plus maze; that is, whereas all CUMS rat groups exhibited stress-associated behaviours prior to intervention, after intervention, the probiotic-fed rats improved these behaviours almost similar to the control rats that had never been exposed to CUMS, and similar to the improvement seen with the addition of fluoxetine.

TABLE 3
Effects of CUMS and Intervention on Total Distance
Travelled (in Metres) in Elevated Maze Test

| | Base line | After CUMS | After intervention |
|---|---|---|---|
| Group | (x̅ ± S) | (x̅ ± S) | (x̅ ± S) |
| Control | 8.96 ± 1.36 | 7.88 ± 1.97 | 7.31 ± 2.41^{b} |
| CUMS | 8.48 ± 2.09 | 3.41 ± 2.67^{a} | 1.97 ± 0.99 |
| CUMS-Flu | 7.44 ± 1.38 | 2.37 ± 1.12^{a} | 6.19 ± 1.75^{b} |
| CUMS-HN001 | 8.28 ± 0.81 | 3.17 ± 1.88^{a} | 8.61 ± 1.63^{b} |
| CUMS-HN019 | 9.35 ± 1.38 | 3.08 ± 2.21^{a} | 7.91 ± 0.65^{b} |
| CUMS- | 8.68 ± 1.28 | 3.37 ± 1.76^{a} | 8.82 ± 1.78^{b} |
| HN001&HN019 | | | |

^{a}Compared to control, p < 0.05;
^{b}Compared to CUMS only, p < 0.05

TABLE 4
Effect of CUMS and Intervention on Time in
Closed Arms (Seconds) in Elevated Plus Maze

| | | | After |
|---|---|---|---|
| | Base line | After CUMS | intervention |
| Group | (x̅ ± S) | (x̅ ± S) | (x̅ ± S) |
| Control | 221.58 ± 29.23 | 232 ± 18.27 | 231.95 ± 19.88^{b} |
| CUMS | 220.70 ± 28.09 | 292.13 ± 7.13^{a} | 285.07 ± 11.58 |
| CUMS-Flu | 240.31 ± 16.95 | 292.57 ± 8.90^{a} | 224.96 ± 18.71^{b} |
| CUMS-HN001 | 230.69 ± 23.78 | 280.00 ± 9.58^{a} | 220.28 ± 11.45^{b} |
| CUMS-HN019 | 215.83 ± 23.30 | 288.83 ± 11.57^{a} | 215.28 ± 17.98^{b} |
| CUMS- | 214.63 ± 26.22 | 289.93 ± 9.96^{a} | 219.80 ± 17.43^{b} |
| HN001&HN019 | | | |

^{a}Compared to control, p < 0.05;
^{b}Compared to CUMS only, p < 0.05

The results of the first and second behavioural tests show that rats exposed to CUMS showed depression-like behaviour and that treatment with HN001 and/or HN019 was able to attenuate this behaviour.

### Example 5: Probiotics Attenuated the Elevated Inflammatory Factors in Serum, Brain and Colon

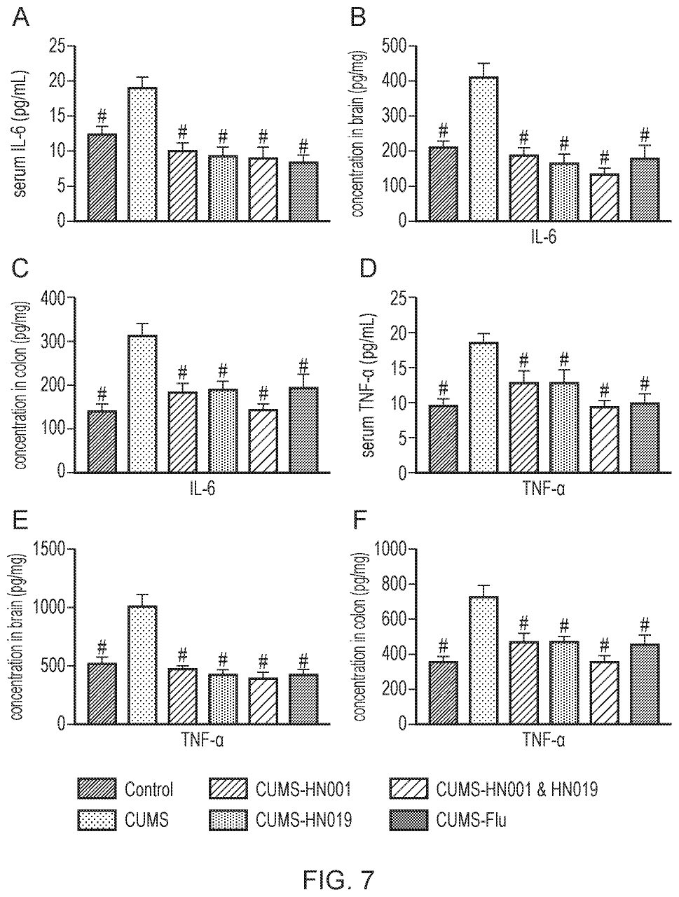

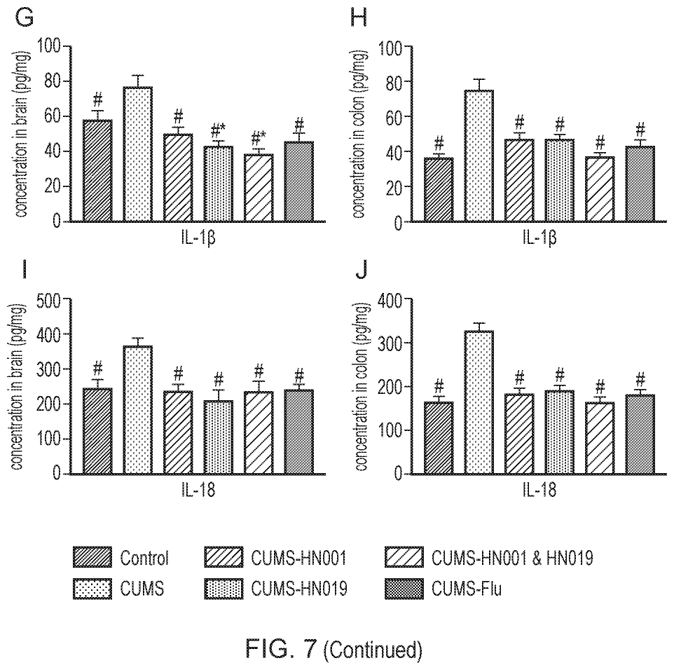

A series of blood markers were assessed. Results indicate that CUMS appeared to increase levels of the cytokines TNF-alpha, IL-6, IL-1β and IL-18 in serum, brain tissue and in the colon compared with controls (P<0.05). Treatment with probiotics and fluoxetine were associated with reduced levels of cytokines (see Tables 5 and 6 below and FIG. 7).

TABLE 5
Comparison of TNF-α Content in Serum, Brain and Colon (pg/ml)

| | serum-after | serum-after | | |
|---|---|---|---|---|
| Group | CUMS | intervention | Brain | Colon |
| Control | 10.51 ± 2.61 | 9.08 ± 3.05^{b} | 57.64 ± 2.27b | 71.73 ± 13.00^{b} |
| CUMS | 19.57 ± 4.83^{a} | 21.60 ± 4.49 | 105.73 ± 7.11 | 100.57 ± 11.88 |
| CUMS-Flu | 23.35 ± 6.46^{a} | 9.83 ± 3.83^{b} | 48.62 ± 7.26^{b} | 69.09 ± 12.48^{b} |
| CUMS-HN001 | 21.12 ± 6.40^{a} | 12.88 ± 4.62^{b} | 61.99 ± 5.17^{b} | 79.89 ± 11.48^{b} |
| CUMS-HN019 | 23.97 ± 3.69^{a} | 12.08 ± 3.68^{b} | 60.67 ± 6.65^{b} | 71.34 ± 10.46^{b} |
| CUMS- | 21.35 ± 4.87^{a} | 10.46 ± 4.81^{b} | 55.16 ± 5.81^{b} | 69.48 ± 16.81^{b} |
| HN001&HN019 | | | | |

^{a}Compared to control, p < 0.05;
^{b}Compared to CUMS only, p < 0.05

TABLE 6
Comparison of IL-6 Content in Serum, Brain and Colon (pg/ml)

| | serum-after | serum-after | | |
|---|---|---|---|---|
| Group | CUMS | intervention | Brain | Colon |
| Control | 9.81 ± 3.38 | 11.67 ± 3.16^{b} | 21.70 ± 2.47^{b} | 29.58 ± 8.80^{b} |
| CUMS | 13.48 ± 4.38^{a} | 17.27 ± 6.22 | 38.32 ± 5.90 | 39.42 ± 8.01 |
| CUMS-Flu | 13.35 ± 6.46^{a} | 9.83 ± 3.83^{b} | 19.35 ± 4.29^{b} | 30.89 ± 7.22^{b} |
| CUMS-HN001 | 14.01 ± 5.98^{a} | 9.96 ± 4.10^{b} | 20.44 ± 3.06v | 31.56 ± 9.00^{b} |
| CUMS-HN019 | 13.08 ± 4.30^{a} | 10.19 ± 4.47^{b} | 19.25 ± 4.78^{b} | 31.88 ± 7.83^{b} |
| CUMS- | 15.39 ± 4.50^{a} | 10.58 ± 5.66^{b} | 17.79 ± 3.33^{b} | 29.18 ± 5.79^{b} |
| HN001&HN019 | | | | |

^{a}Compared to control, p < 0.05;
^{b}Compared to CUMS only, p < 0.05

### Example 6: Correlation Analysis of Inflammatory Factors in Brain and Colon

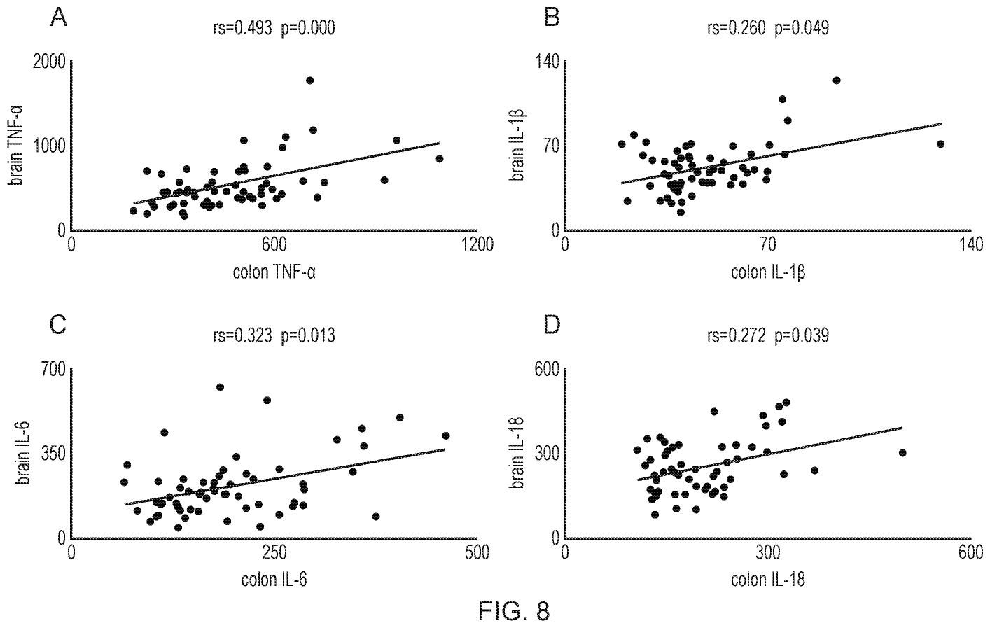

To further verify that intestinal inflammation is linked to brain inflammation, the correlation between intestinal inflammation factors and those in the brain was assessed. A clear positive correlation between TNF-α in brain and colon was found (rs=0.493, p=0.000). A positive correlation between IL-6 (rs=0.323, p=0.013), IL-1 (rs=0.260, p=0.049) and IL-18 (rs=0.272, p=0.039) in brain and colon was also found. See FIG. 8.

### Example 7: Effect on Neuroendocrine Hormones—Corticotropin Releasing Hormone (CRH) and Adrenocorticotropic Hormone (ACTH) Content

Testing of elements of the HPA axis showed that like Fluoxetine, treatment with HN001, HN019, or HN001+HN019 was able to reduce levels of certain neuroendocrine hormones and hormone receptors induced stress following CUMS treatment. See Table 7 and 8 below.

TABLE 7
Comparison of Serum Corticotropin Releasing
Hormone (CRH) Content (pg/ml)

| Group | After CUMS | After intervention | P |
|---|---|---|---|
| Control | 2.84 ± 1.28 | 3.00 ± 1.61^{b} | 0.705 |
| CUMS | 5.77 ± 3.19^{a} | 5.25 ± 2.30 | 0.498 |
| CUMS-Flu | 5.50 ± 2.02^{a} | 2.03 ± 1.27^{b} | 0.000 |
| CUMS-HN001 | 5.53 ± 2.77^{a} | 2.38 ± 1.59^{b} | 0.012 |
| CUMS-HN019 | 5.19 ± 3.48^{a} | 2.47 ± 1.34^{b} | 0.024 |
| CUMS- | 5.45 ± 2.64^{a} | 1.83 ± 1.35^{b} | 0.001 |
| HN001&HN019 | | | |

^{a}Compared to control, p < 0.05;
^{b}Compared to CUMS only, p < 0.05

TABLE 8
Comparison of Serum Adrenocorticotropic
Hormone (ACTH) Content (pg/ml)

| Group | After CUMS | After intervention | P |
|---|---|---|---|
| Control | 2.46 ± 1.77 | 2.93 ± 2.10^{b} | 0.157 |
| CUMS | 5.29 ± 2.36^{a} | 5.84 ± 2.95 | 0.567 |
| CUMS-Flu | 5.58 ± 1.41^{a} | 2.90 ± 2.20^{b} | 0.013 |
| CUMS-HN001 | 5.85 ± 3.35^{a} | 3.12 ± 2.52^{b} | 0.011 |
| CUMS-HN019 | 5.53 ± 3.43^{a} | 2.13 ± 1.88^{b} | 0.009 |
| CUMS- | 5.27 ± 3.43^{a} | 3.23 ± 2.27^{b} | 0.044 |
| HN001&HN019 | | | |

^{a}Compared to control, p < 0.05;
^{b}Compared to CUMS only, p < 0.05

### Example 8: Western Blot Analysis of Neuroendocrine Hormone Receptors—Corticotropin Releasino Factor Type 1 (CRF1) and Glucocorticoid Receptor (GR) in Brain Tissue

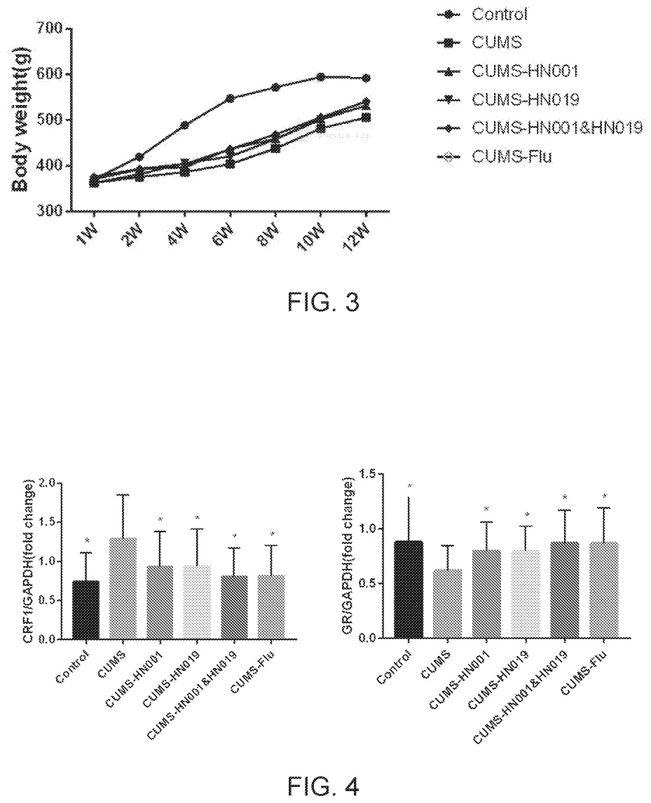

Western blot analysis results showed that like Fluoxetine, consumption of both or either of the probiotic strains could reverse CUMS-induced changes in HPA axis-associated neuroendocrine receptor expression in the brain. Results are shown in FIG. 4 as ratios of antibody staining intensity of receptor (CFR1, left; GR, right) vs the housekeeping protein GAPDH. * P<0.05.

### Example 9: Probiotics Normalised the Morphological Changes of Hippocampi and Colon

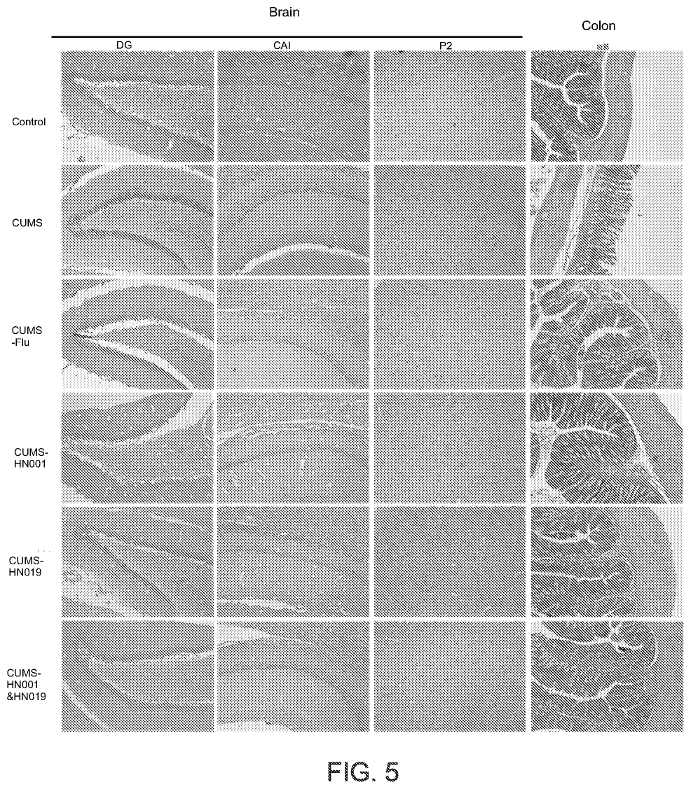

Brain and colon tissue were stained using the HE method. Brain tissue included two regions from the hippocampus (Dentate Gyrus (DG) and CA1) and a region from the cortex (P2). The results are shown in FIG. 5 and reveal that for the control group, neurons in hippocampal regions (dentate gyrus (DG) and Ca1) and the cortex (P2) were normal, orderly, tightly packed cells. The cells had expected morphology, homogeneous cytoplasmic staining, and no increased interstitial or abnormal changes. In contrast, corresponding tissues from CUMS rats showed disordered, irregular shape of some cells, pyknosis of nuclei, unclear nucleoli, and uneven cytoplasmic staining. However, tissues from all four intervention groups (Fluoxetine, HN001, HN019, and HN001+HN019) showed that the CUMS-associated damage was largely reduced.

For the colon tissue, control intestinal villi were arranged neatly, with normal cell morphology and no obvious damage was seen. Compared with the control group, the intestinal villi of the CUMS group were disordered, and showed obvious damage. Thickness of the intestinal mucosa was significantly thinner than that of the control group. This CUMS-associated damage was again largely reduced by consumption of the probiotic strains, similar to Fluoxetine.

### Example 10: Effects of Probiotics on Neurotransmitters and their Metabolites in Serum and Brain

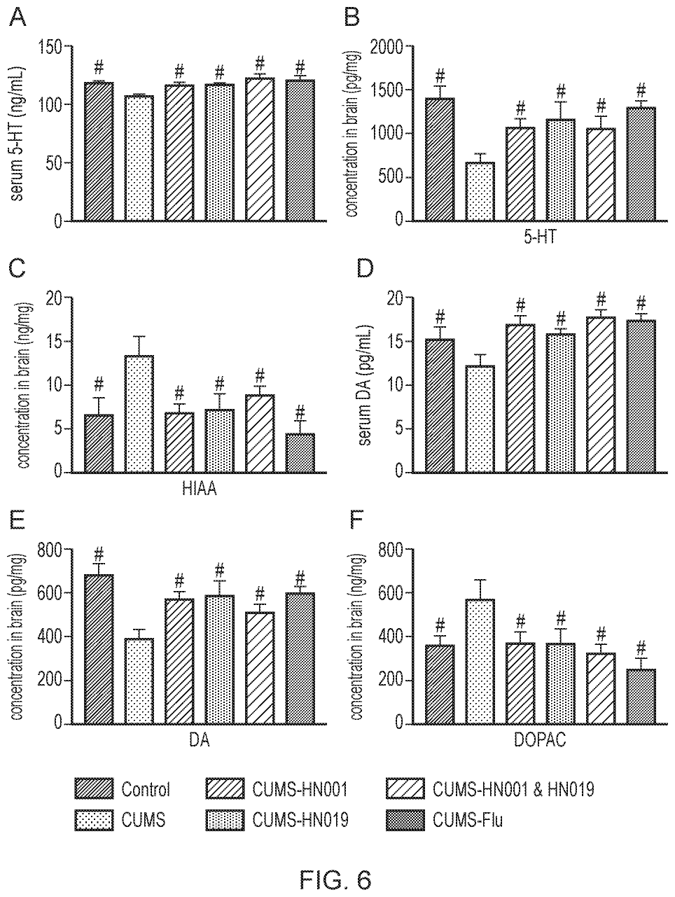

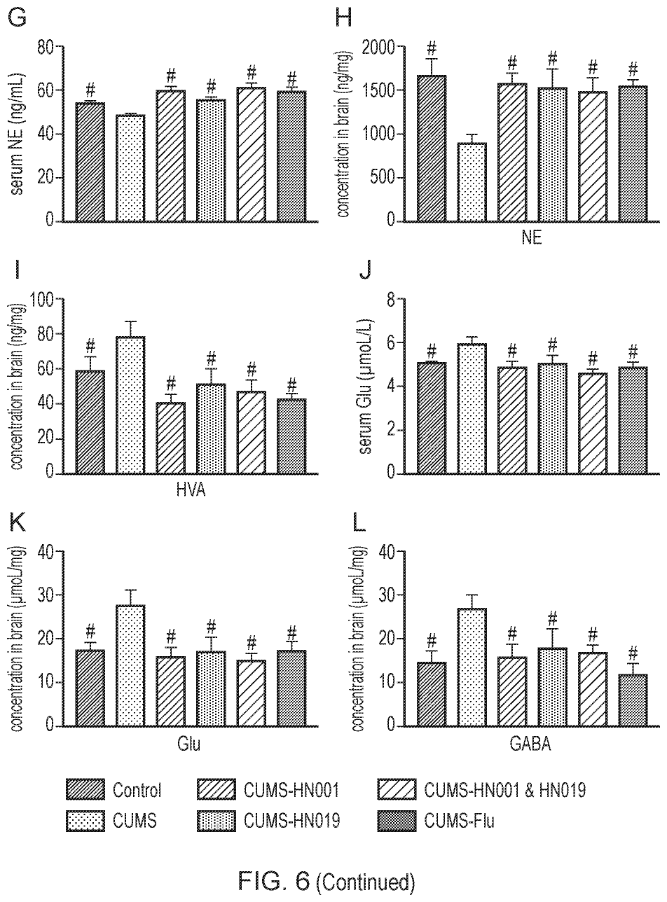

CUMS significantly decreased the content of 5-HT, DA, NE and increased the concentration of Glu and GABA in serum and brain (FIG. 6 A-L). HIAA, DOPAC and HVA, as the metabolites of 5-HT, DA and NE, were increased in rat brains after CUMS stimulation. Administration with HN001 and/or HN019 was able to reverse the effects of CUMS on these neurotransmitters.

### Example 11: Alterations of Microbiota Induced by Probiotic Treatment

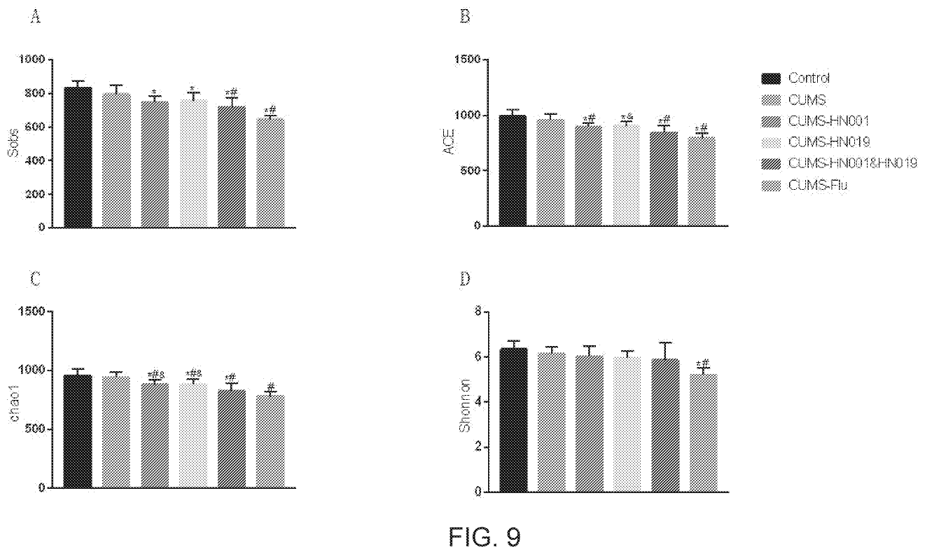

To evaluate whether probiotics, as well as CUMS, alter intestinal microbiota, 16S rRNA sequencing of faecal samples was performed. Alpha-diversity, as measured by richness (Sobs index, Chao1 index and ACE index; see FIGS. 9 A-C), and diversity (Shannon index; see FIG. 9 D), was decreased after CUMS.

Interestingly, compared with CUMS group, Sobs, ACE, Chao1 and Shannon index were decreased in other groups administered with HN001 and/or HN019. Moreover, the Shannon Index in combined group was significantly decreased compared with that of the single group, which means that the diversity of intestinal microbiota changed markedly. The change in the dominant microbiota of the intestine after intervention may explain the results. Further analysis determined the dominant flora related to anxiety and depression-like behaviours.

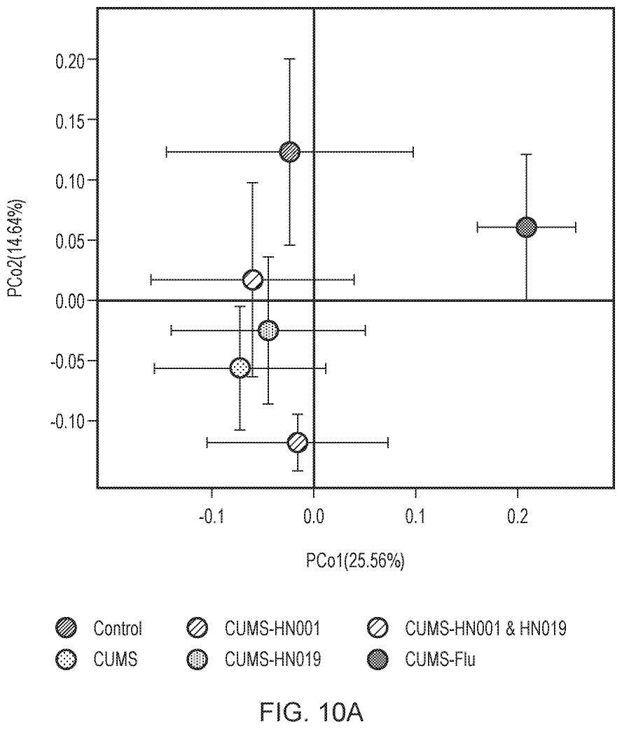

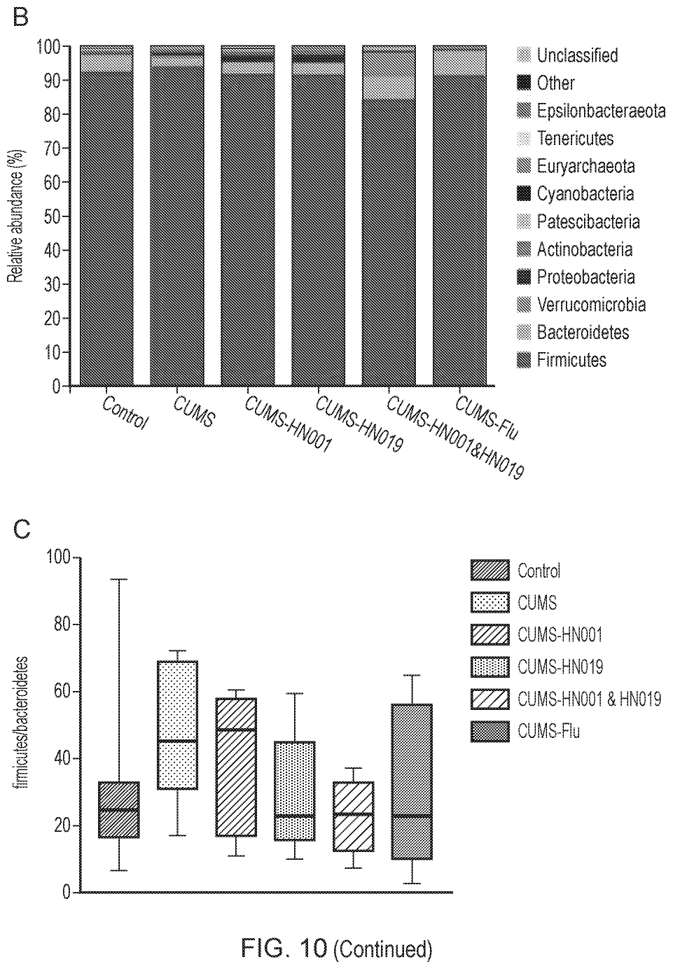

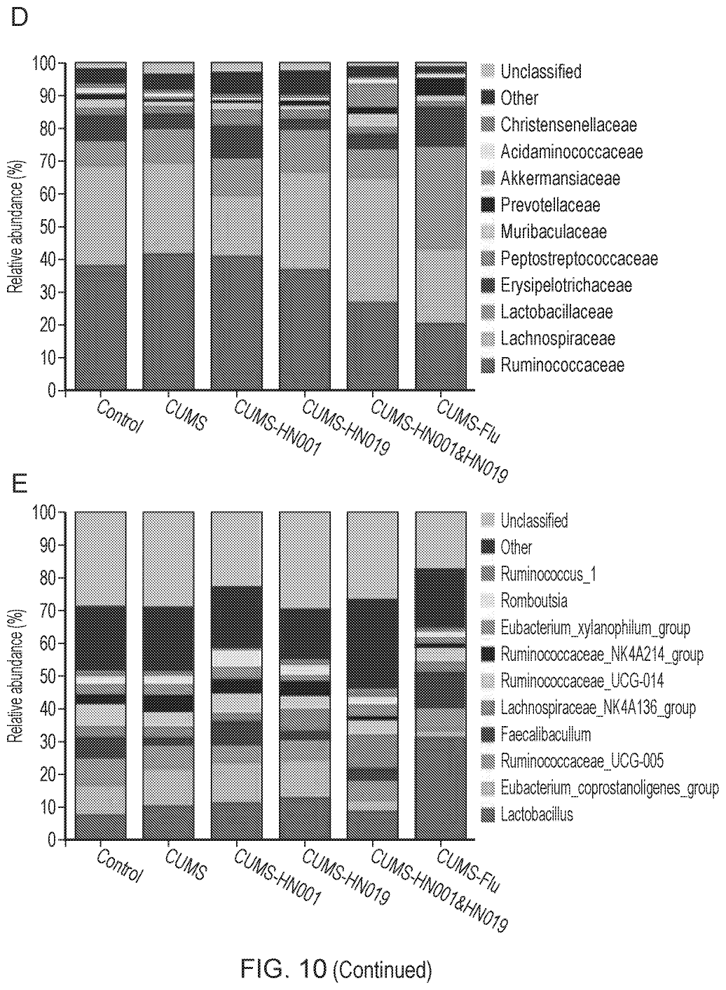

Beta diversity analysis was evaluated through PCoA to assess differences in microbial composition among six groups (FIG. 10A). The PCoA results showed that the microbial communities in the CUMS group exhibited a distinct composition compared to that of control rats. Moreover, HN001, HN019 and HN001 & HN019 treatment induced marked differences in the PCoA plot in comparison with those of CUMS rats. Especially the gut microbiota profiles of the HN001 & HN019-treated rats demonstrated a significant separation from those of CUMS rats.

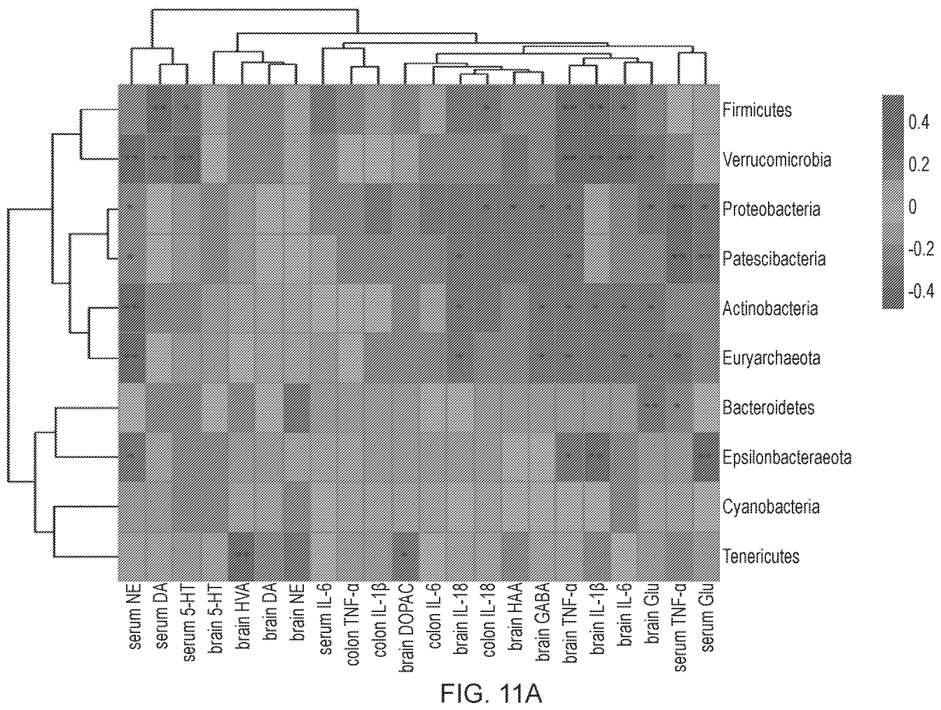

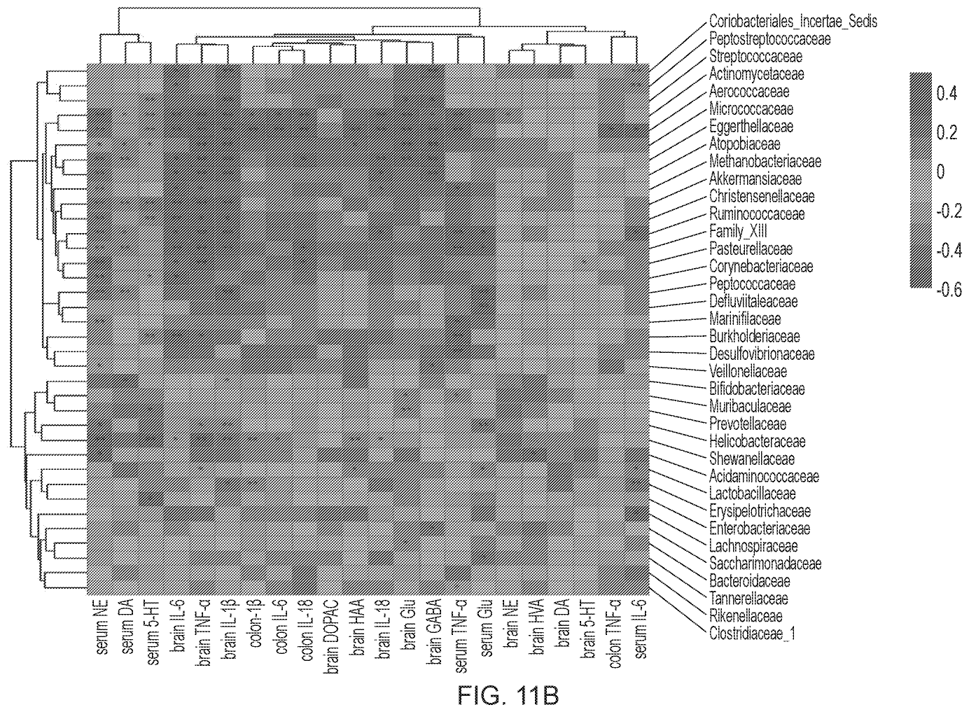

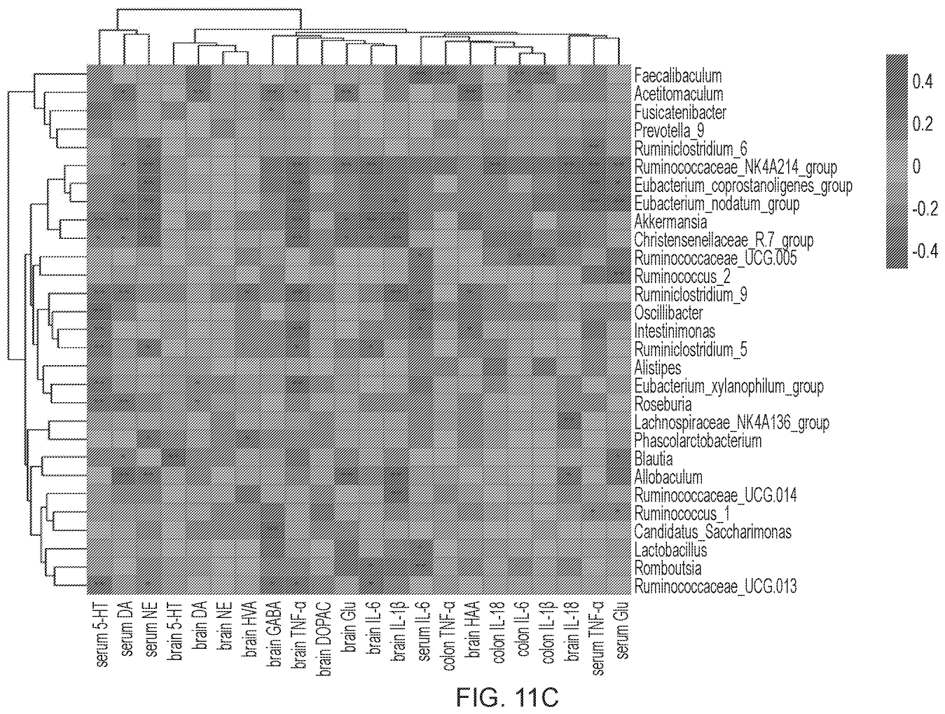

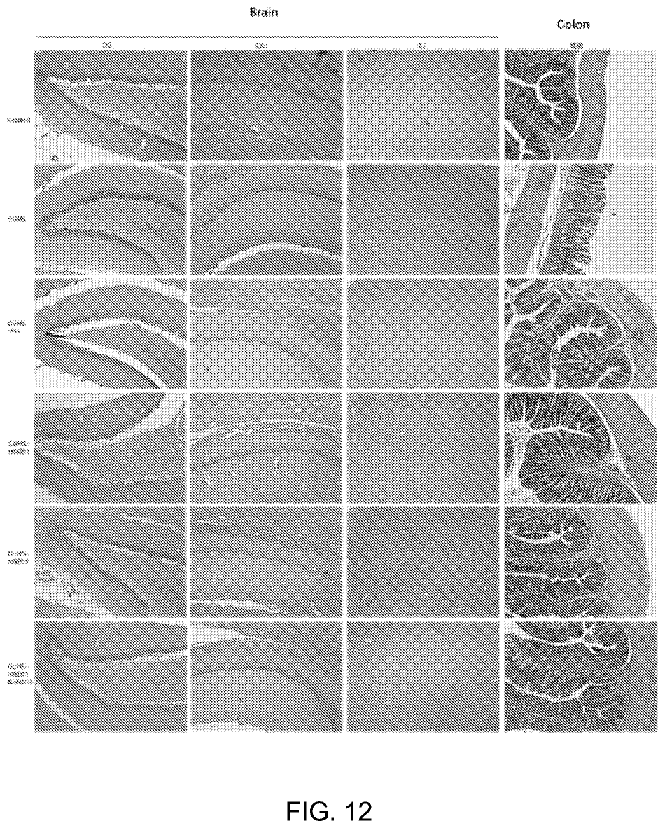

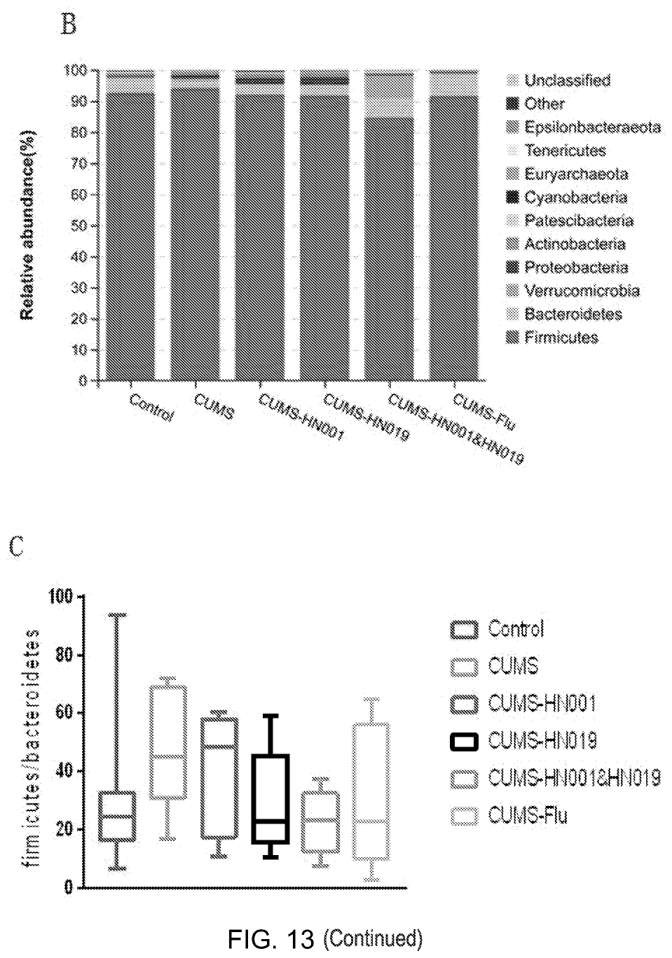

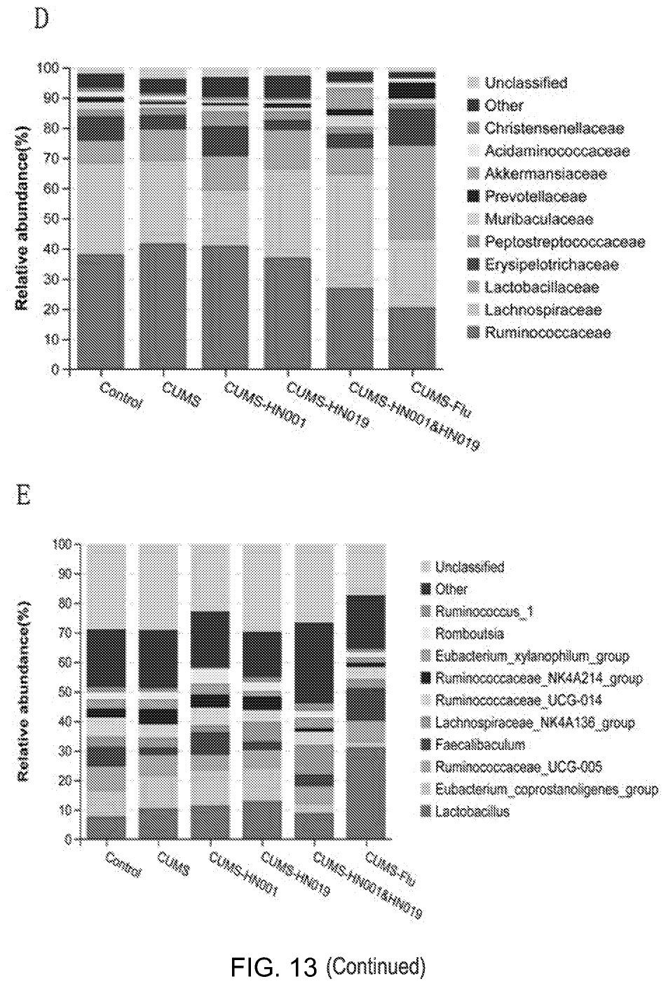

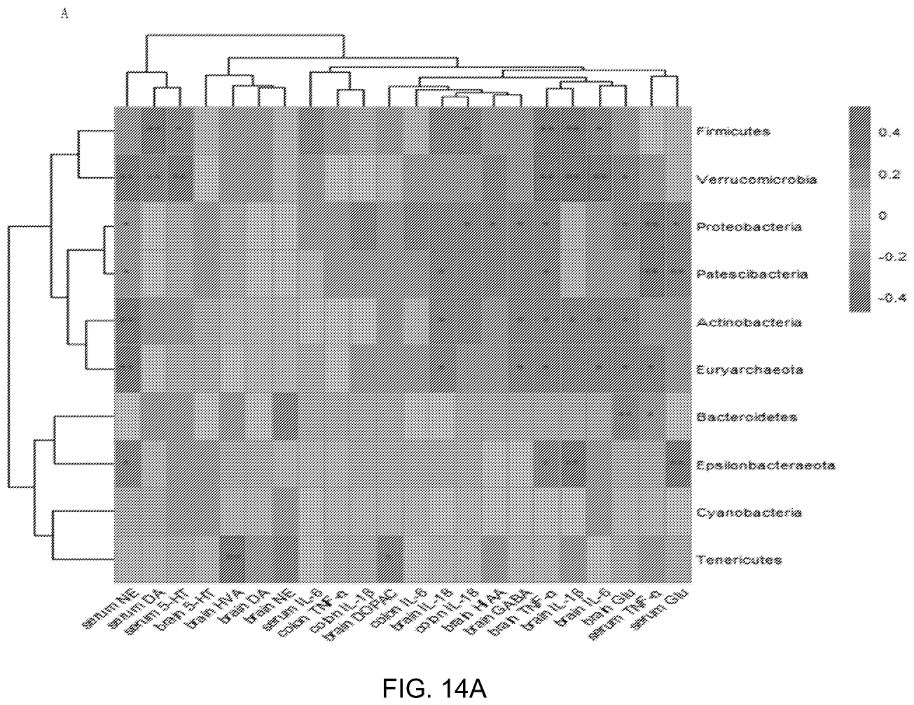

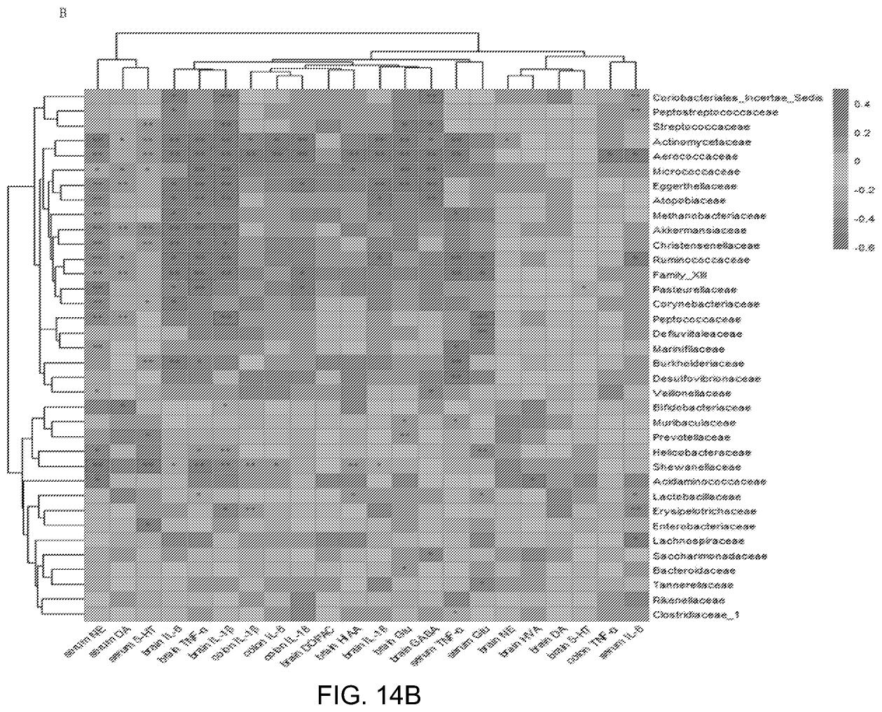

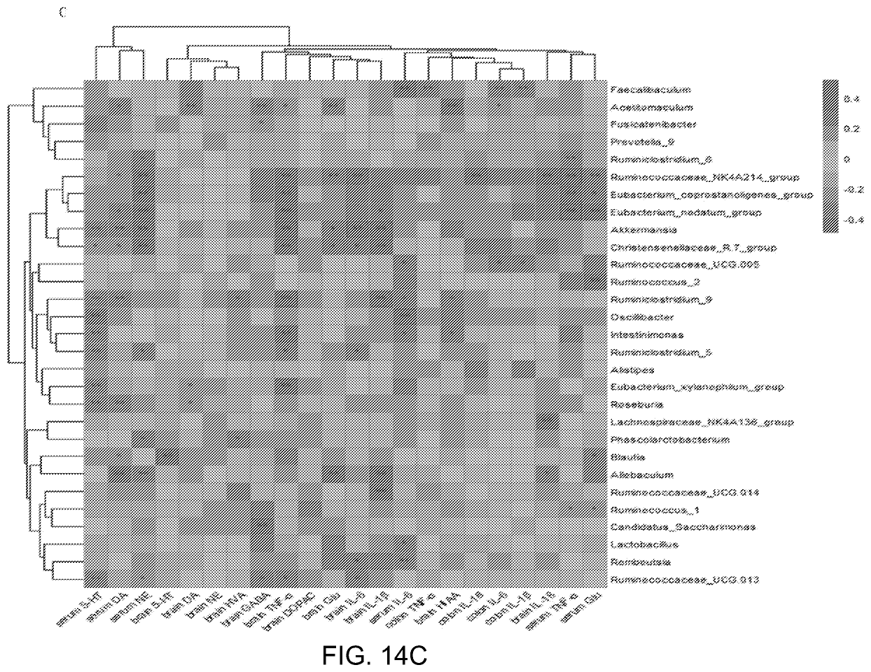

According to the species abundance cluster heat map, at phylum level (FIG. 11B), Firmicutes, Bacteroidetes, Verrucomicrobias, Proteobacterias and Actinobacterias were the dominant phyla. The relative abundance is shown in FIG. 10. CUMS downregulated the relative abundance of Bacteroidetes, while upregulated the relative abundance of the other bacteria mentioned above. Treatment with HN001, HN019 and HN001 & HN019 alleviated the phylum alterations, and the changes of bacteria in the combined group was more marked. Meanwhile, after CUMS stimulation, Firmicutes to Bacteroidetes ratio (F/B ratio) was increased and administration with HN001 and/or HN019 decreased the F/B ratio.

At the family level, the relative abundance of Ruminococcaceae and Lactobacillaceae was elevated, while the Lachnospiraceae were declined in the CUMS group, and HN001 and/or HN019 reversed the changes of family, especially the joint intervention. Genera Lactobacillus, Eubacterium coprostanoligenes group, and Lachnospiraceae-NK4A136-group, all of which belong to the phylum Firmicutes, were enhanced and decreased respectively, and these changes were also improved by HN001 and/or HN019 supplementation. The relative ratio of microbiota was changed after CUMS treatment and HN001 and/or HN019.

Correlation Analysis of Intestinal Microbiota with Monoamine Neurotransmitters and Inflammatory Factors

Monoamine neurotransmitter deficiency and inflammatory factor imbalance are important in the pathogenesis of depression. To elucidate the associations between changed microbiota and corresponding depression related traits including neurotransmitters and inflammatory factors, Spearman's correlation analysis was performed. As shown in FIG. 9, Firmicutes, Verrucomicrobia, Proteobacteria, Pastescibacteria, Actinobacteria and Euryarchaeota had positive correlations with inflammatory factors in serum, brain and colon, and negative correlations with the level of NE, DA and 5-HT in the serum. On the other hand, Epsilonbacteraeota and Bacteroidetes exhibited positive correlations neurotransmitters concentrations in serum, and negative correlations with inflammatory mediators. Otherwise, clear correlations could be identified between the family and genera microbiota and these indicators. In particular, family Lachnospiraceae, Ruminococcaceae, Christensenellaceae and Peptostreptococ-caceae and genera Ruminococcaceae, Eubacterium_coprostanoligenes_group, Ruminococcaceae_UCG014, Ruminoco-ccaceae_NK4A214_group and and Ruminococcus-1, all of which belong to Firmicutes, showed positive associations and negative associations with inflammatory factors and serum neurotransmitters respectively. Muribaculaceae and Prevotellaceae under the Bacteroidetes were negative related with inflammatory factors, and showed positive correlation with 5-HT, NE, DA in serum, which were consistent with Bacteroidetes. These results indicated that altered microbiota, especially certain microbiota, which were specifically affected by HN001 and (or) HN019, showed strong associations with neurotransmitters and inflammatory cytokines simultaneously, demonstrating the critical role of gut microbiota in regulating neurotransmitters and inflammation.

Together, these results show that probiotic HN001 and HN019, alone or together, can reduce stress and anxiety in a rat model, via the HPA axis. Interestingly, the probiotics also seemed to have anti-inflammatory effects, in that probiotic consumption was associated with reduced TNF-alpha and IL-6 cytokine levels that were increased in CUMS rats without probiotic treatment. Also, the probiotics were able to reverse the impacts of CUMS on brain and colon cell morphology, protecting these tissues from cell damage associated with stress.

Compared to pharmaceutical treatments of mild stress and anxiety, probiotics generally are without side effects, and can be delivered via a wide range of dairy and non-dairy foods as well as supplements. Current experiments show similar effects as an SSRI, commonly prescribed to combat conditions such as anxiety, obsessive compulsive disorder or post-traumatic stress disorder.

Throughout the description and claims of this specification, the words “comprise” and “contain” and variations of the words, for example “comprising” and “comprises”, mean “including but not limited to”, and do not exclude other components, integers or steps. Moreover the singular encompasses the plural unless the context otherwise requires: in particular, where the indefinite article is used, the specification is to be understood as contemplating plurality as well as singularity, unless the context requires otherwise.

Preferred features of each aspect of the invention may be as described in connection with any of the other aspects. Within the scope of this application it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

1. (canceled)

2. A composition comprising:
a. Lactocaseibacillus rhamnosus strain HN001 (HN001) or derivatives thereof, and/or
b. Bifidobacterium animalis subsp. lactis strain Hn019 (HN019) or derivatives thereof, and
c. a physiologically acceptable diluent, adjuvant, carrier or excipient.

3. A method for any one or more of: (i) reducing or preventing stress, optionally chronic stress; (ii) reducing or preventing anxiety; (iii) reducing or preventing one or more symptoms of depression; (iv) decreasing or reversing changes caused by stress, anxiety or depression; (v) improving mood; (vi) increasing relaxation; (vii) increasing energy, said method comprising administering a therapeutically effective amount of Lacticaseibacillus rhamnosus strain HN001 (HN001) and/or Bifidobacterium animalis subsp. lactis strain HN019 (HN019) or derivatives thereof to a subject in need thereof, with the proviso that HN001 is not used in a method of treating or preventing postnatal depression, postnatal anxiety or stress in a subject.

4. The method according to claim 3, wherein decreasing or reversing changes caused by stress, anxiety or depression include any one or more of the following:
a) changes in levels of neurotransmitters or their metabolites,
b) changes in levels of stress hormones
c) changes in levels of inflammation factors,
d) morphological changes to brain tissue,
e) morphological changes to the colon,
f) changes to gut microbiota, and
g) changes in weight.

5. The method according to claim 3, wherein said HN001 and/or HN019 or derivatives thereof are administered orally to a subject in need.

6. The method according to claim 3, wherein said administering reduces levels of one or more molecules involved in the hypothalamic-pituitary-adrenal (HPA) axis, such as levels of any one or more of the following: corticotrophin-releasing hormone (CRH), adrenocorticotrophic hormone (ACTG), and levels of glucocorticoid hormones, such as cortisol; and/or optionally wherein said reduced levels are in any one or more of serum, brain or intestinal tissues.

7. The method according to claim 3, wherein said administering reduces inflammation, optionally as manifested through a reduction in inflammatory cytokines, such as TNF-alpha, IL-6, IL-1β, IL-18; and/or optionally wherein said reduced inflammation is in any one or more of serum, brain or intestinal tissues.

8. (canceled)

9. The method according to claim 3, wherein said administering of HN001 and/or HN019 or derivatives reverses stress-induced decreases in any one or more of serotonin (5-HT), dopamine (DA), and norepinephrine (NE) or metabolites thereof and/or increases the concentration of glutamate and/or GABA to levels found in non-stressed individuals.

10. The method according to claim 3, wherein said administering of HN001 and/or HN019 or derivatives restores changes in gut microbiota caused by stress back to their non-stress profile, which may be in terms of abundance, (alpha and beta) diversity, richness, levels of dominant flora etc.

11. The method according to claim 3, wherein said administering of HN001 and/or HN019 or derivatives thereof reverses the impact of stress and/or anxiety on brain or colon cell morphology and/or protects brain or colon tissue from cell damage associated with stress and/or anxiety; and/or optionally wherein said administering of HN001 and/or HN019 or derivatives thereof protects and maintains epithelial cell barriers from damage and loss of integrity, for example, due to inflammation.

12. (canceled)

13. The method according to claim 3, wherein said administering of HN001 and/or HN019 or derivatives thereof protects or improves Blood Brain Barrier (BBB) integrity, optionally leading to reduced brain inflammation and/or improved brain function; and/or optionally wherein said administering of HN001 and/or HN019 or derivatives thereof protects or improves gut barrier integrity, optionally wherein said use is in the treatment or prevention of irritable bowel syndrome (IBS) and/or systemic inflammation.

14-15. (canceled)

16. The method according to claim 3, wherein the reducing stress and/or anxiety occurs via the HPA axis.

17. The method according to claim 3, wherein the reducing stress comprises any one or more of: reduced stomach ache, muscle tension, headaches, rapid breathing, shortness of breath, fast heartbeat, palpitations, sweating, dry mouth, shaking, fatigue, fidgeting, accelerated speech, augmentation of negative emotions (if already being experienced), longer duration of stress fatigue, change in appetite, dizziness, confusion, feelings of impending doom, panic, nervousness, difficulty in concentrating, gut discomfort or disfunction, irrational anger and restlessness.

18. The method according to claim 3, wherein the reducing anxiety comprises a reduction in apprehension and feelings of impending danger, catastrophe, misfortune, unease, worry, nervousness, and fear or apprehension around the subject's current or future state.

19. The composition according to claim 2, wherein HN001 is represented by Genebank accession number NZ_ABWJ00000000 and is available under AGAL deposit number NM97/09514 dated 18 Aug. 1997 and wherein the HN019 is available under AGAL deposit number NM97/09513.

20. The composition according to claim 19, wherein HN001, HN019 or derivatives thereof are in a reproductively viable form and/or are killed, lysed, fractionated or attenuated.

21. The method according claim 3, wherein HN001 and/or HN019 or derivatives thereof are administered in a food product or supplement.

22. The method according to claim 21, wherein the food product or supplement further comprises further probiotic(s) and/or prebiotic(s).

23. The method according to claim 21, wherein the food product is selected from: cultured milk, yoghurt, cheese, milk drink and milk powder.

24. The method according to claim 21, wherein the food product is selected from a maternal supplement, a dietetic product, an infant formula, a follow-on formula, and a growing-up formula.

25. The method according to claim 21, wherein the food product or supplement is administered daily to provide at least about 1.9×10⁹ cfu of HN001 and/or 6.5×10⁷ cfu of HN019 per day to establish a population in the gastrointestinal tract of human subjects.
